(19) United States
(12) Patent Application Publication
(10) Pub. No.: US 2008/0160031 A1
(43) Pub. Date: Jul. 3, 2008

(54) Method and Composition for Treatment of Neoplasms

(75) Inventor: Darren Raymond Shafren, Newcastle (AU)

Correspondence Address: SEED INTELLECTUAL PROPERTY LAW GROUP PLLC, 701 FIFTH AVE, SUITE 5400, SEATTLE, WA, 98104, US

(73) Assignee: Viralytics Limited, Pymble (AU)

(21) Appl. No.: 11/795,439

(22) PCT Filed: Jan. 17, 2006
(86) PCT No.: PCT/AU06/00051

§ 371 Date: Feb. 26, 2008

(30) Foreign Application Priority Data
Jan. 17, 2005 (AU) 2005900179

(51) Int. Cl. A61K 39/395; A61K 31/7052; A61P 35/00

(52) U.S. Cl. 424/155.1; 514/44

(57) ABSTRACT

The invention relates to methods of treating a neoplasm in an animal, in particular treating a neoplasm in a human, through the use of isolated nucleic acid sequence, including synthetic viral RNA and complementary DNA, derived from one or more Picornaviruses. The invention also relates to compositions of isolated nucleic acids derived from one or more Picornaviruses, and to the use of isolated nucleic acids derived from one or more Picornaviruses for the manufacture of a medicament for the treatment of neoplasms in a mammal.

### FIELD OF THE INVENTION

The present invention relates to methods of treating a neoplasm in an animal, in particular treating a neoplasm in a human, through the use of isolated nucleic acid sequence, including synthetic viral RNA and complementary DNA, derived from one or more Picornaviruses. The invention also relates to compositions of isolated nucleic acids derived from one or more Picornaviruses, and to the use of isolated nucleic acids derived from one or more Picornaviruses for the manufacture of a medicament for the treatment of neoplasms in a mammal.

### BACKGROUND

Viral oncolytic therapy is emerging as a promising treatment for a number of human and animal cancers. For example Shafren et al (2004; “Systemic Therapy of Malignant Human Melanoma Tumors by a Common Cold-Producing Enterovirus, Coxsackievirus A21”. Clin Cancer Res. 10(1):53-60) demonstrated the efficiency of the oncolytic capacity of Coxsackievirus serotype CVA21 in vivo using a human melanoma NOD-SCID mouse model. Immuno-compromised mice bearing established subcutaneous melanoma xenografts, treated with an intra-tumoural (i.t.) dose of CVA21 exhibited significantly reduced tumour burdens compared to phosphate buffered saline (PBS) treated controls. Intra-peritoneal (i.p.) and intravenous (i.v.) routes of virus administration of CVA21 were also demonstrated to be effective in reducing melanoma xenograft burden.

The specificity of a particular virus to target and lyse a susceptible cancerous cell whilst avoiding non-malignant, normal cells, presents advantages this therapy compared to conventional treatments, such as radiotherapy and chemotherapy. Characteristic changes in cell surface receptor expression and alterations in cell signaling pathways, associated with malignant transformation of a cell, are utilized by oncolytic viruses to distinguish cancerous cells from normal cells. PCT/AU2003/001688 (published as WO/2004/054613 entitled “A method of treating a malignancy in a subject via direct picornaviral-mediated oncolysis”), for example, describes the use of Echoviruses, such as Echovirus serotypes EV1, EV7, EV8 and EV22, for cell destruction of a variety of cancer cell types, such as breast, colorectal, prostate, ovarian and melanoma cells. Cellular recognition may be used to advantage, for example Echovirus serotypes EV1 and EV8 which recognize the cellular receptor α₂β₁ for infectivity of a cell, and EV7 and EV22 which recognize the complement regulatory protein decay accelerating factor (DAF) for infectivity of a cell. Similarly, Picornaviruses that recognize at least one of a cell adhesion molecule, such as intercellular adhesion molecule-1 (ICAM-1) and a complementary regulatory protein, such as DAF, have also been shown to be capable of targeted destruction of cancer cells, such as melanoma, breast and prostate cancer cells. As demonstrated in PCT/AU00/01461 (published as WO01/37866 and entitled “A method of treating a malignancy in a subject and a pharmaceutical composition for use in same”), such Picornaviruses include Coxsackieviruses such as Coxsackievirus serotypes CVA13, CVA15, CVA18 and CVA21.

Following infection, an oncolytic virus can kill a cancerous cell by direct lytic infection, induction of apoptosis or by initiating an immune response to viral antigens. An oncolytic virus is thus not limited to a single input dose and can undergo a multi-cycle infection, resulting in the production of large numbers of progeny virus. These progeny can spread either locally to adjacent tumour cells, or systemically to distant metastatic sites. This feature of oncolytic therapy is particularly attractive for the treatment of inaccessible tumours or un-diagnosed micro-metastases.

### Malignant Melanoma

Malignant melanoma is a tumour derived from activated or genetically altered epidermal melanocytes. Minor populations of melanocytes within the skin (basal epidermis), eye, hair and mucous membranes normally function to pigment the skin/hair by producing and distributing melanin to keratinocytes. An array of complex interactions between genetic and environmental factors, are known to induce malignant transformation of melanocytes, including genetic predisposition and exposure to environmental ultra-violet irradiation.

Malignant melanoma progresses through a number of defined stages. The initial, localized lesion (nevi) usually exhibits a radial growth phase (RGP), restricted to the epidermis. From here, the melanoma progresses into a vertical growth phase (VGP) incorporating the dermis, and developing into an expansive tumour nodule with metastatic capabilities. The progression of a melanoma into this metastatic mode has a significant negative impact on patient survival.

Current therapies for malignant melanoma include surgical excision, radiotherapy, chemotherapy and biotherapies all of which have a relatively low success rate (approximately 1 in 14) (Russell-Jones, R. and K. Acland, Sentinel node biopsy in the management of malignant melanoma. Clin Exp Dermatol, 2001. 26(6):463-468; Slominski A, et al, Malignant Melanoma: An Update, Archives of Pathology and Laboratory Medicine, 2001. 125(10):1295-1306. If diagnosed before metastasis occurs, surgical excision can often provide an effective, long-term treatment. However, following cellular metastasis, the lesion becomes malignant, and current therapies in particular chemotherapy offer little, to no therapeutic benefit (Atkins, M. B., The treatment of metastatic melanoma with chemotherapy and biologics. Curr Opin Oncol, 1997. 9(2): p. 205-13).

The need for an effective, specific treatment is becoming more apparent with the increasing incidence of melanoma worldwide. Australia has one of the highest incidence rates of melanoma, with over 8500 new cases being diagnosed in the year 2000 (Cancer in Australia 2000, in AIHW (Cancer Series no. 23). 2000, Australian Institute of Health and Welfare (AIHW) & Australasian Association of Cancer Registries (AACR) 2003: Canberra: p. AIHW cat. no. CAN 18). In New South Wales alone, over 3000 new cases and 425 deaths were reported in 2002, and the incidence rates per 100 000 were the third highest in the world, surpassed only by Queensland and Western Australia (Tracey E A, et al, Cancer in New South Wales: Incidence and Mortality 2002. 2004, The Cancer Council NSW, New South Wales Department of Health: Sydney). For these reasons, a number of novel therapeutics for melanoma have been proposed.

Malignant melanoma cells express high levels of the complement regulatory protein, decay accelerating factor (DAF) and cellular adhesion molecule, intercellular adhesion molecule type 1 (ICAM-1). The increased expression of these molecules affords the tumour cell a number of attributes beneficial to its long-term survival. Increased DAF expression allows cancerous cells to evade complement-mediated degradation (Cheung, N. K., et al., Decay-accelerating factor protects human tumor cells from complement-mediated cytotoxicity in vitro. J Clin Invest, 1988. 81(4):1122-8), and up-regulated ICAM-1 expression is postulated to increase the metastatic potential of a tumour by facilitating interaction with invasive lymphocytes (Johnson, J. P., et al., De novo Expression of Intercellular-Adhesion Molecule 1 in Melanoma Correlates with Increased Risk of Metastasis. Proc Natl Acad Sci USA, 1989. 86(2):641-644). Increases in tumour thickness (VGP) correlate to increased expression of ICAM-1, and greater metastatic capability. Metastatic cells expressing high surface levels of ICAM-1 characteristically secrete high levels of soluble ICAM-1 (sICAM-1) into the circulation, levels of which, are used as a prognostic factor for malignant melanoma progression (Vuoristo, M.-S., et al., Serum adhesion molecules and interleukin-2 receptor as markers of tumour load and prognosis in advanced cutaneous melanoma. European Journal of Cancer, 2001. 37(13):1629-1634).

The up-regulation of both ICAM-1 and DAF normally allows a melanoma cell to undergo uncontrolled proliferation and metastasis, however, this characteristic has recently been exploited for the targeted viral oncolysis of tumour cells both in vitro and in vivo. CVA21, a member of the Picornaviridae family, was recently shown to specifically target and lyse melanoma cells exhibiting up-regulation of both ICAM-1 and DAF Shafren et al 2004; ibid).

### The Picornaviridae

The Picornaviridae is one of the largest families of viruses named using the Greek ‘pico’ (very small), and ‘RNA’ after their ribonucleic acid genome. The family contains a number of clinically significant human and animal pathogens including poliovirus, is rhinovirus and hepatitis A. The Picornaviridae family is divided into nine genera based on physical virion properties, RNA sequence similarities and viral RNA genomic organization (Table 1.2) (Stanway, G., et al., Molecular and Biological Basis of Picornavirus Taxonomy, in Molecular Biology of Picornaviruses, in B. Semler and E. Wimmer, Editors. 2002, ASM Press: Washington D.C. p. 17-24).

TABLE 1
Picornaviridae Genera. The nine genera of the Picornaviridae
family of RNA viruses are shown. Representative
species for each genus are listed.

| Genus | Representative Species |
|---|---|
| Enterovirus | Poliovirus |
| | Coxsackievirus |
| | Echovirus |
| Rhinovirus | Human Rhinovirus |
| Cardiovirus | Encephalomyocarditis virus |
| Aphthovirus | Foot and Mouth Disease virus |
| Hepatovirus | Hepatitis A virus |
| Parechovirus | Human Parechovirus |
| Teschovirus | Porcine Teschovirus |
| Erbovirus | Equine rhinitis B virus |
| Kobovirus | Aichi virus |

Overall, the above findings suggest that CVA21 and other Picornaviruses are potential virotherapy agents for the control of malignant melanoma and other cancers. Therapies based on preparation and administration of live virus may raise community concerns such as bio-safety issues associated with the production, distribution and administration of infectious virus. Alternative methods for the treatment of malignant melanoma and other neoplasms utilizing oncolytic viral therapy may obviate these perceptions.

### SUMMARY OF THE INVENTION

In a first aspect the present invention provides a method for treating a neoplasm in a mammal requiring said treatment, the method comprising administering to the mammal an effective amount of a nucleic acid molecule comprising an isolated viral polynucleotide sequence derived from a Picornavirus under conditions which result in virus-mediated oncolysis of one or more cells of the neoplasm.

In one embodiment the neoplasm is selected from the group consisting of prostate cancer, breast cancer, ovarian cancer, lymphoid cancer, leukemia, brain cancer, lung cancer, colorectal cancer, thyroid cancer, renal cancer, adrenal cancer, liver cancer, stomach cancer, intestinal cancer and melanoma.

In one embodiment the nucleic acid molecule may be selected from single stranded RNA or complementary DNA (cDNA) comprising a sequence derived from the Picornavirus.

In one embodiment the sequence may constitute an entire viral genome or a portion thereof capable of eliciting a lytic infection when administered to a cell.

In one embodiment the nucleic acid molecule is synthetic viral RNA.

In one embodiment the nucleic acid molecule is derived from a Picornavirus which recognises at least one of a cell adhesion molecule of the immunoglobulin superfamily and a complement regulatory protein for infectivity of a cell.

In one embodiment the nucleic acid molecule is derived from a Picornavirus which recognises α₂β₁ for infectivity of a cell.

In one embodiment the nucleic acid molecule is derived from a Picornavirus which recognises at least one of ICAM-1 and DAF for infectivity of a cell.

In one embodiment the nucleic acid molecule is derived from a Picornavirus capable of lyrically infecting or inducing apoptosis in a cell substantially in the absence of ICAM-1.

In one embodiment the nucleic acid molecule is derived from a Picornavirus capable of lytically infecting or inducing apoptosis in a cell through DAF on the cell.

In one embodiment the nucleic acid molecule is derived from a Coxsackie virus selected from the group consisting of CVA13, CVA15, CVA18 and CVA21.

In one embodiment the nucleic acid molecule is derived from an Echovirus selected from the group consisting of EV1, EV7, EV8 and EV22.

In one embodiment the polynucleotide sequence comprises an alteration in one or more capsid proteins compared with wild-type wherein the alteration enhances cell selectivity and or neoplasm targeting of a virus comprising the alteration.

In one embodiment the polynucleotide sequence comprises a Coxsackie virus nucleic acid sequence comprising one or more mutations of a coat protein.

In one embodiment the mutation(s) of the coat protein comprises one or more mutation(s) selected from the group consisting of VP3 R96H, VP3 E101A, VP3 A239S, VP2 S164L and VP2 V209 or corresponding conservative variants thereof.

In one embodiment the nucleic acid molecule is administered as a formulation comprising vRNA and lipid, such as a cationic lipid.

In one embodiment the formulation further comprises a ligand which recognises and interacts with a tumour-specific marker.

In one embodiment the formulation further comprises an antibody which recognises a tumour antigen, such as a monoclonal antibody which recognises DAF, ICAM-1, α₂β₁, or MAGE.

In one embodiment the nucleic acid molecule is administered by direct injection into a neoplasm.

In one embodiment the nucleic acid molecule is administered orally or systemically.

In one embodiment the method further comprises administration of one or more immunosuppressants to the mammal.

In one embodiment the mammal is a human.

In a second aspect the invention provides a method for treating melanoma in a human requiring said treatment, the method comprising administering to the human an effective amount of a vRNA:lipid formulation, wherein the vRNA comprises RNA isolated from one or more viruses selected from the group consisting of CVA13, CVA15, CVA18, CVA21, EV1, EV7, EV8 and EV22 variants CVA21 #272101, CVA21 #275238, and CVA21 #272598, and CVA21-DAFv, under conditions which result in virus-mediated oncolysis of one or more cells of the melanoma.

In one embodiment the administration is direct injection of the formulation into one or more melanomas of the human.

In one embodiment the formulation further comprises a ligand which recognises and interacts with a tumour-specific marker.

In one embodiment the formulation further comprises an antibody which recognises a tumour antigen, such as a monoclonal antibody which recognises DAF, ICAM-1, α₂β₁, or MAGE.

In a third aspect the invention provides use of a nucleic acid molecule comprising an isolated viral nucleic acid sequence derived from a Picornavirus for the preparation of a medicament for the treatment of a neoplasm in a mammal.

In a fourth aspect of the invention there is provided a pharmaceutical composition comprising a nucleic acid molecule comprising an isolated viral polynucleotide sequence derived from a Picornavirus together with a pharmaceutically acceptable vehicle, diluent or carrier, wherein administration of the pharmaceutical composition to a neoplasm results in virus-mediated oncolysis of one or more cells of the neoplasm.

In one embodiment the nucleic acid molecule is derived from one or more viruses selected from the group consisting of CVA13, CVA15, CVA18, CVA21, EV1, EV7, EV8 and EV22 variants CVA21 #272101, CVA21 #275238, and CVA21 #272598, and CVA21-DAFv.

In one embodiment the composition comprises one or more lipids, such as a cationic lipid.

In one embodiment the composition comprises a vRNA:lipid complex, such as a CVA21:lipid complex.

In one embodiment the composition comprises a ligand which recognises and interacts with a tumour-specific marker.

In one embodiment the formulation further comprises an antibody which recognises a tumour antigen, such as a monoclonal antibody which recognises DAF, ICAM-1, α₂β₁, or MAGE.

### ABBREVIATIONS

CD cluster of differentiation
cDNA complementary DNA
° C. degrees Celsius
CHO Chinese hamster ovary
CPE cytopathic effect
CSPD disodium 3-(4-methoxyspiro{1,2-dioxetane-3,2′-(5chloro)tricyclo[3.3.1.1^{3,7}]decan}-4-yl)phenyl phosphate
CVA21 Coxsackievirus A21
DAF decay accelerating factor
DIG digoxigenin-11-2′-deoxy-uridine-5′-triphosphate
DMEM Dulbecco's modified Eagle's Medium
DNA deoxyribonucleic acid
FCS fetal calf serum
GPI glycosylphosphatidylinositol
ICAM-1 intercellular adhesion molecule type 1
Ig immunoglobulin
IgSF immunoglobulin super family
i.p. intra-peritoneal
IRES internal ribosome entry site
i.t. intra-tumoural
i.v. intra-venous
kDa kilo dalton
LFA-1 leukocyte function-associated antigen 1
MAb monoclonal antibody
Mac-1 macrophage-1 antigen
mRNA messenger RNA
NOD-SCID non-obese diabetic severe combined immune deficient
PBS phosphate buffered saline
PI post infection
RD rhabdomyosarcoma
RGP radial growth phase
RNA ribonucleic acid
RT room temperature
S.C. subcutaneous
SCR short consensus repeat
TCID₅₀ 50% tissue culture infectious dose
UTR untranslated region
VGP vertical growth phase
vRNA viral RNA
vRNA:lipid viral RNA/lipid complex
VP viral protein
VPg viral protein genome linked

### DEFINITIONS

The following are some definitions that may be helpful in understanding the description of the present invention. These are intended as general definitions and should in no way limit the scope of the present invention to those terms alone, but are put forth for a better understanding of the following description.

In the context of this specification, the term “treatment” refers to any and all uses which remedy a disease state or symptoms, prevent the establishment of disease, or otherwise prevent, hinder, retard, or reverse the progression of disease or other undesirable symptoms in any way whatsoever.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Any description of prior art documents herein, or statements herein derived from or based on those documents, is not an admission that the documents or derived statements are part of the common general knowledge of the relevant art in Australia or elsewhere.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

### BRIEF DESCRIPTION OF THE FIGURES

FIG. 1. Flow Cytometric Analysis of ICAM-1 and DAF surface expression on SK-MeI-28, RD and CHO cells. The black histogram shows binding of the conjugate only; the blue histogram represents binding of anti-ICAM-1 monoclonal antibody (MAb); and the red histogram represents binding of anti-DAF MAb.

FIG. 2. Denaturing Agarose Gel Electrophoresis and Northern Blot of CVA21 RNA. A. The 420-nucleotide 3′ region of CVA21 RNA complementary to the DIG-11UTP labelled DNA probe sequence. B. Two samples of CVA21 RNA (5 μl and 10 μl) and a 0.3-6.9 kb RNA marker (M) were separated on a denaturing 1% agarose gel. C. RNA bands were transferred and fixed to a nylon membrane via Northern Blot capillary transfer, hybridised with a labelled DNA probe, detected with anti-DIG-Alkaline-Phosphatase and CSPD and finally exposed to X-ray film.

FIG. 3. Cell Toxicity of Lipofectamine 2000™ on SK-MeI-28, RD and CHO cells. Monolayers of SK-MeI-28, RD and CHO cells were incubated in 24-well plates with varying concentrations of Lipofectamine 2000™ for 24 hours, then examined microscopically for cell death. High concentrations of Lipofectamine 2000™, 10 μl/well (A) and 5 μl/well (B) were highly toxic to SK-MeI-28 cells, while 2 μl/well was the highest concentration of Lipofectamine 2000™ tolerated by all three cells lines; SK-MeI-28 (C), RD (D), and CHO (E).

FIG. 4. Progression of Viral Cytopathic Effect in Sk-MeI-28 Cells Following Lipid-Mediated Transfection with CVA21 Viral RNA. Monolayers of SK-MeI-28 cells in 24-well plates were transfected with CVA21 vRNA complexed with Lipofectamine 2000™ (vRNA:lipid Complex). Control wells included 2 μl/well Lipofectamine 2000™ only (lipid only), 1 μg/well viral RNA only (vRNA only), and 1.6×10⁴ TCID₅₀/well live CVA21 virions (CVA21 live virus). Cells were monitored for signs of viral cytopathic effect (CPE) for 48 hours post transfection. Photomicrographs display representative sections of each well after 12, 24 and 48 hours. Approximate CPE values observed are shown from no CPE (−), 25% of cells displaying CPE (+), 50% CPE (++), 75% CPE (+++) to 100% (++++). Fifty percent tissue culture infectious doses per ml (TCID₅₀/ml) of well supernatants, determined by lytic cell infectivity assays in monolayers cultures of SK-MeI-28 cells are shown in the bottom left corner of each picture.

FIG. 5. Infectivity Assays of Cell Supernatants Following Transfection with CVA21 vRNA:lipid Complex. Monolayers of SK-MeI-28 cell in 96-well plates were inoculated with 10-fold dilutions of transfection supernatants, at each time-point of interest, from all of the four treatments; CVA21 vRNA:lipid complex (vRNA:lipid), CVA21 vRNA only (vRNA only), lipid only, and CVA21 live virions (CVA21 live). Samples taken at 12 hours post-transfection are shown in this example. Monolayers were incubated for 72 hours at 37° C. in 5% CO₂, microscopically examined for CVA21 induced CPE and stained with crystal violet solution. Wells are marled by plus symbol (+) for CPE positive well. The ratio of CPE positive:CPE negative wells per dilution were used to calculate the 50% tissue culture infectious dose (TCID₅₀) using the method of Reed and Muench (A simple method for estimating fifty percent endpoints. Am. J. Hyg., 1938. 27: p. 493-497).

FIG. 6. Progression of CVA21 Induced Cytopathic Effect and Production of Progeny Virus in RD and CHO Cells Following Transfection with CVA21 vRNA. Monolayers of RD (A) and CHO (B) cells in 24-wells plates were transfected with vRNA complexed with Lipofectamine 2000™ (vRNA:lipid). Control wells were inoculated with an initial dose of 2 μl per well of Lipofectamine 2000™. Photomicrographs display sections of each well following 12, 24 and 48 hours incubation. Approximate CPE values observed are shown; no CPE (−), 25% of cells displaying CPE (+), 50% (++), 75% (+++) and 100% (++++). Fifty percent tissue culture infectious doses per ml,(TCID₅₀/ml) of well supernatants, determined by lytic cell infectivity assays in monolayers cultures of SK-MeI-28 cells are shown in the bottom left corner of each picture.

FIG. 7. Development of CVA21 Induced Cytopathic Effect in RD and CHO Cells Following Passage of vRNA:lipid Transfection Supernatants. Supernatants harvested at 48 hours following transfection of RD and CHO cells were passaged onto monolayers of RD or CHO cells in 24-well plates to examine the infectivity of progeny infectious CVA21 on these cells. No CPE was observed in either cell type following 48 hours incubation at 37° C. in 5% CO₂.

FIG. 8. Real-time RT-PCR of CVA21 Viral RNA Extracted from Serum of NOD-SCID Mice Bearing Established Subcutaneous Melanoma Xenografts Injected with vRNA:lipid Complex or CVA21 Live Virus. RNA extracted from serum samples was screened for CVA21 vRNA. Threshold level (red line) was set within the linear region of the exponential phase of amplification when plotted as log of change in fluorescence (Log Delta Rn) against cycle number. Samples with fluorescence exceeding this threshold were positive for CVA21 vRNA and viral titres were calculated by comparing the threshold cycle (CT) of these unknowns to the CT value of standard CVA21 preparations of known titre (STD 10³-10⁷ TCID₅₀/ml). Each mouse sample is identified by the cage number (C1-4) and number of ear tags (one left 1L, one right 1R, one left and one right LR, two right 2R, no holes NH).

FIG. 9. Reduction in Tumour Volume of Established Human Melanoma Xenografts in NOD-SCID Mice Treated with CVA21 vRNA:lipid Complex or CVA21 Live Virus. A. Four groups of NOD-SCID mice bearing established subcutaneous SK-MeI-28 human melanoma tumours were injected intra-tumourally with one of four treatments; CVA21 vRNA:lipid Complex (vRNA:lipid, n=5), vRNA only (n=4), lipid only (n=4) or CVA21 live virus (n=4) on days 0 and 8. Mice treated with CVA21 live virus were not injected on day 8 as real-time RT-PCR screening reported high titres of CVA21 in the sera of this group. B. Two intersecting measurements of each tumour were used to calculate tumour volume (mm³) using the formula for the volume of spheroid. Average values for each group are shown from day 0 (day of initial treatment) to day 35, beyond which, sacrifice of a number of mice for ethical reasons limited the statistical relevance of group average values. Error bars indicate standard error (SD/√n). * Statistically significant compared to lipid only treated group (p<0.05).

FIG. 10. Post Mortem Tumour Burden Examination of NOD-SCID Mice Bearing Subcutaneous Melanoma Xenografts Treated with Lipid only, CVA21 vRNA only, vRNA:lipid Complex, or CVA21 Live Virus. Mice bearing established subcutaneous SK-MeI-28 human melanoma xenografts were treated intra-tumourally with A. lipid (Lipid only) or B. CVA21 vRNA (vRNA only) on two occasions, day 0 and day 8. Forty four days following the initial treatment, mice were sacrificed and tumours exposed by the removal of fur and skin from the back of each mouse. Mice treated on two occasions with C. CVA21 vRNA:lipid complex (vRNA:lipid) on days 0 and 8 or one treatment of D. CVA21 live virus on day 0. Tumours were exposed post mortem by the removal of fur and skin. Serum viral load, reported as 50% endpoint titres (TCID₅₀/ml) was tested by real-time RT-PCR(PCR) and cell infectivity assays (Infect. Assay). Positive CVA21 viremia is represented by a plus sign (+), negative viremia by a minus sign (−). Sections of each tumour were excised, homogenised and tumour viral load (TCID₅₀/μg) tested by cell infectivity assays of homogenate supernatants. Two tumours were undetectable on examination (Not Detectable) therefore no tumour viral load is shown.

### DETAILED DESCRIPTION

Although it is known that some naturally occurring and modified Picornaviruses, such as Coxsackievirus and Echovirus, are suitable for use in treatment of cancer, the present inventor has identified a need for additional methods for treatment of cancer, such as methods which offer an alternative or an adjunct to the administration of live virus.

The present invention arises from the observation and demonstration by the inventor herein that administration of isolated Picornavirus nucleic acid to abnormal cells, as demonstrated herein by melanoma, is capable of eliciting a productive viral infection in the cells, thereby leading to cell destruction.

Accordingly, the invention provides a method for treating a neoplasm in a mammal requiring said treatment, the method comprising administering to the mammal an effective amount of a nucleic acid molecule comprising an isolated viral polynucleotide sequence derived from a Picornavirus under conditions which result in virus-mediated oncolysis of one or more cells of the neoplasm.

The nucleic acid molecule may be administered to the cell as an isolated nucleic acid. It will be appreciated that the term “isolated” includes polynucleotide sequence that has been derived from a Picornavirus including, for example, a nucleic acid sequence encoding the viral genome or a sufficient sequence thereof to permit generation of the virus or to be capable of eliciting a lytic infection in a cell. Thus, the nucleic acid molecule may comprise a single viral RNA or DNA molecule, such as a complementary DNA molecule, or a plurality of such molecules encoding different viral sequences.

The term “polynucleotide” as used herein refers to a single- or double-stranded polymer of deoxyribonucleotide, ribonucleotide bases or known analogues or natural nucleotides, or mixtures thereof.

It is to be understood that in the context of the specification the term “derived” from thus includes that the sequence may be viral RNA directly isolated from the Picornavirus, synthetic RNA, cDNA corresponding to the isolated sequence. The term also includes synthetic polynucleotide sequences comprising one or more mutations in the sequence compared to wild-type sequence, including, for example mutations in the capsid proteins.

Neoplastic cells may be transfected with viral RNA extracted from purified virions or for instance RNA transcripts may be generated in vitro from cDNA templates utilizing bacteriophage T7 RNA polymerase as described in Ansardi, D. C. et al., 2001. Similarly, a single plasmid or RNA molecule may be administered for expression of viral proteins and generation of virus, or a plurality of plasmids or RNA molecules encoding different ones of the viral proteins may be administered for transfecting the cells and generation of the virus.

Any suitable method for isolation of viral RNA may be used, including methods based on the use of phenol/chloroform extraction, such as provided in commercial kit form for isolation of viral RNA, such as Trizol® LS reagent (GIBCO BRL, Life Technologies Grand Island, N.Y., USA), isolation methods which utilize magnetic bead-based isolation, such as Ambion MagMax™ viral RNA isolation kits. Methods for the isolation of viral RNA are generally described in, for example Ausubel, F., et al., eds. Current Protocols in Molecular Biology. 1992, Green Publishing Associates and Wiley-Interscience, John Wiley and Sons: New York. and in Sambrook et al., (1989), Molecular Cloning: A Laboratory Manual, Second Ed., Cold Spring Harbour Laboratory Press, New York.

It will be appreciated that the method does not require the viral RNA, whether it be directly isolated from virus, synthesized, presented as a plasmid molecule or generated in vitro such as from cDNA templates using bacteriophage T7 RNA polymerase, to be devoid of contaminant material, such as cell debris, to be considered “isolated” in the context of this specification. Thus, in the context of the specification RNA will be considered isolated when non-RNA components from the source material, such as cellular proteins, have been partially or completely removed from the RNA. For example, the RNA will be considered “isolated” when greater than 50% of non-RNA material has been removed. It is preferred that greater than 60% of the non-RNA material be removed, more preferably greater than 70%, 80% or 90% of the non-RNA material will be removed. Typically, the RNA will contain less than 10% contaminant material, more typically less than 5% contaminant material. Thus, the RNA will preferably be greater than 95% pure for viral RNA, even more preferably greater than 97% pure or greater than 99% pure.

Rather than administration of viral RNA per se, viral or other plasmids or expression vectors incorporating nucleic acid for generation of the virus may be injected into the tumor (neoplasm) for uptake by tumor cells and generation of intact virus within the cells for effecting the cell death. Suitable expression vectors include plasmids capable of expression of a DNA (eg. Genomic DNA or cDNA) insert encoding viral proteins necessary for generation of the virus. An expression vector will typically include transcriptional regulatory control sequences to which the inserted nucleic acid is operably linked. By “operably linked” is meant the nucleic acid insert is linked to the transcriptional regulatory control sequences for permitting transcription of the inserted sequence(s) without a shift in the reading frame of the insert. Such transcriptional regulatory control sequences include promotions for facilitating binding of RNA polymerase to initiate transcription, and expression control elements for enabling binding of ribosomes to transcribed mRNA.

More particularly, the term “regulatory control sequence” as used herein is to be taken to encompass any DNA that is involved in driving transcription and controlling (ie. regulating) the level of transcription of a given DNA sequence. For example, a 5′ regulatory control sequence is a DNA sequence located upstream of a coding sequence and which may comprise the promotor and the 5′ untranslated leader sequence. A 3′ regulatory control sequence is a DNA sequence located downstream of the coding sequence(s), which may comprise suitable transcription termination (and/or) regulation signals, including one or more polyadenylation signals. As used herein, the term “promotor” encompasses any DNA sequence which is recognized and bound (directly or indirectly) by a DNA-dependant RNA polymerase during initiation of transcription. A promotor includes the transcription initiation site, and binding sites for transcription initiation factors and RNA polymerase, and can comprise various other sites or sequences (eg. enhances), to which gene expression regulatory proteins may bind.

Numerous expression vectors suitable for transfection of mammalian cells are known in the art. Expression vectors suitable for transfection of mammalian cells include pSV2neo, pEF-PGk.puro, pTk2 and non-replicating adenoviral shuttle vectors incorporating the polyadenlation site and elongation factor 1-x promotor and pAdEasy based expression vectors most preferably incorporating a cytomegalovirus (CMV) promotor (eg. See He et al., 1998). The plasmid pEFBOS which employs the polypeptide elongation factor—alpha 2 as the promotor may also be utilized.

cDNA encoding the viral proteins necessary for generation of the virus may be prepared by reverse transcribing the viral RNA genome or fragments thereof and incorporated into a suitable vector utilizing recombinant techniques well known in the art as described in for example Sambrook et al., (1989), Molecular Cloning: A Laboratory Manual, Second Ed., Cold Spring Harbour Laboratory Press, New York, and Ausubel et al., (1994) Current Protocols in Molecular Biology, USA, Vol. 1 and 2.

Plasmids or RNA may be administered directly to tumors either topically or by injection for uptake by the tumor cells in the absence of a carrier vehicle for facilitating transfection of the cells or in combination with such a vehicle.

Suitable carrier vehicles include liposomes typically provided as an oil-in-water emulsion conventionally known in the art. Synthetic lipid vesicles (liposomes) facilitate the delivery of various molecules, including nucleic acids across the cell membrane. Liposomes have been utilized for the delivery of nucleic acids, cytotoxic drugs, and even cosmetics, to cells both in vitro and in vivo. Recent advances in liposome technologies have improved their efficiency for nucleic acid delivery. Cationic liposomes exhibiting a net positive charge, are the most widely used type. These liposomes function by either encapsulating or complexing a negatively charged nucleic acid, allowing it to overcome the repulsive electrostatic forces between it and the cell membrane (also negatively charged). If encapsulated, the lipid acts as a synthetic membrane, surrounding the nucleic acid molecule. If in a complex, the lipid carries the nucleic acid on its outer surface. The molecule being carried is then taken up by a target cell either by fusion of the membranes and expulsion of the liposome contents, or by endocytosis of the entire complex. Several commercially available cationic liposomes have proven successful for both in vitro and in vivo transfection of eukaryotic cells (Audouy S, H. D., Cationic Lipid-mediated Transfection in vitro and in vivo (Review). Molecular Membrane Biology, 2001. 18:129-143; Dalby, B., S. et al., Advanced transfection with Lipofectamine 2000 reagent: primary neurons, siRNA, and high-throughput applications. Methods, 2004. 33(2):95-103; Egilmez, N. K., et al., Evaluation and Optimization of Different Cationic Liposome Formulations for in Vivo Gene Transfer. Biochemical and Biophysical Research Communications, 1996. 221(1):169-173; Reynier P, et al., In Vitro and In Vivo transfection of Melanoma Cells B16-F10 mediated by Cholesterol-based Cationic Liposomes. J Drug Targeting, 2002. 10(7):557-566).

Liposomes will typically comprise a combination of lipids, particularly phospholipids such as high phase transition temperature phospholipids usually with one or more steroids or steroid precursors such as cholesterol for providing membrane stability to the liposomes. Examples of lipids useful for providing liposomes include phosphatidyl compounds such as phosphatidylglycerol, phosphatidylcholine, phosphatidylserine, sphingolipids, phosphatidylethanolamine, cerebrosides and gangliosides. Diacyl phosphatidylglycerols are particularly suitable, where the lipid moiety contains from 14 to 18 carbon atoms and more preferably from 16 to 18 carbon atoms, and is saturated. Commercial sources of liposomes suitable for the present invention are available and include, for example, Lipofectamine 2000 (Invitrogen Life Technologies, Carlsbad Calif., USA). Appropriate and optimal concentrations of liposome and liposome:nucleic acid may be determined by the skilled addressee using methods known in the art and methods described herein and, for commercial sources of liposomes, methods described by the manufacturer.

Interaction of the liposomes with the target cells may be passive or active. Active targeting involves modification of the liposome by incorporating in the liposome membrane a specified ligand which binds or otherwise interacts with the corresponding ligand expressed by the target cells. Such ligands include for example a monoclonal antibody or binding fragment thereof (eg. an Fab or F(ab′)₂) fragment, a sugar or glycolipid moiety, or a viral protein, viral proteins or monoclonal antibodies specific for α₂β₁, ICAM-1 or DAF are particularly preferred, as are monoclonal antibodies or other ligands specific for melanoma antigen-encoding (MAGE) gene products, for example as described in Chen Z, et al., Expression of A, G and B melanoma antigen genes in human hepatocellular carcinoma. Hepatobiliary Pancreat Dis Int. 2002 November; 1(4):570-3. The use of such targeting ligands may be preferred when the nucleic acid molecule is administered orally or systemically.

The nucleic acid sequence may be derived from a naturally occurring Picornavirus, or from a modified Picornavirus, such as may be prepared by intentional or unintentional bioselection or recombinant methods.

In the context of this specification the term “naturally occurring” Picornavirus will be understood to mean a Picornavirus that can be isolated from a source in nature and which has not been intentionally modified by humans in the laboratory.

The Picornavirus the source of the nucleic acid may be a Picornavirus modified by recombinant means such as are known in the art and described, for example, in Ausubel et al and in Sambrook et al. Alternatively or in addition the Picornavirus may be modified by bioselection such as described by Johansson et al (2004; J. Virol. 78(22): 12603-12612.

In one embodiment the Picornavirus is selected from the group consisting of Coxsackievirus, Echovirus and modified forms thereof.

As described in PCT/AU00/01461, published as WO 01/37866, entitled “A method of treating a malignancy in a subject and a pharmaceutical composition for use in same” viruses of the Picornaviridae family that recognize at least one of a cell adhesion molecule, such as ICAM-1, and a complement regulatory protein, such as DAF, are capable of killing abnormal cells, such as cancer cells. The contents of PCT/AU00/01461 are incorporated herein by cross-reference.

Accordingly it will be appreciated that the Picornavirus nucleic acid molecule of the present invention may be that of a Picornavirus that recognizes at least one of a cell adhesion molecule, such as ICAM-1, and a complement adhesion molecule, such as DAF, for infectivity of a cell. For example the Picornavirus may be a Coxsackievirus, such as a Coxsackie A-group virus, for example one or more of Coxsackie A-group virus serotypes CAV1 through CAV21.

In one embodiment the Coxsackie A-group virus is selected from the group consisting of CAV13, CAV15, CAV18 and CAV21.

As described in co-pending application PCT/AU2003/001688 (published as WO/2004/054613 and entitled “A method of treating a malignancy in a subject via direct picornaviral-mediated oncolysis”), Echoviruses which recognize α₂β₁ for infectivity of cells are capable of inducing cell lysis and so may be used for treatment of abnormal cells, such as cancer cells, in a mammal. Accordingly it will be appreciated that the Picornavirus nucleic acid of the present invention may be that of an Echovirus that recognizes α₂β₁ for infectivity of a cell. For example, the Echovirus may be an Echovirus selected from the group consisting of Echovirus EV1 and EV8. It will also be appreciated that the Picornavirus nucleic acid of the present invention may be that of an Echovirus that recognizes DAF for infectivity of a cell, such as EV7 or EV22. The contents of PCT/AU2003/001688 are incorporated herein by cross-reference.

As noted above the Picornavirus may be a modified Picornavirus produced, for example, by recombinant methods or bioselection methods. For example, co-pending application PCT/AU2005/000048 filed 17 Jan. 2005, published as WO/2005/087931 and entitled “Modified oncolytic viruses” describes, inter alia, methods for the preparation of isolated selected Picornavirus capable of lytic infection or induction of apoptosis of neoplasms. PCT/AU2005/000048 also describes methods for bioselecting a Picornavirus capable of lytically infecting a cell substantially in the absence of intercellular adhesion molecule-1 (ICAM-1). The contents of PCT/AU2005/000048 are incorporated herein by cross-reference.

PCT/AU2005/000048 includes specific examples of bioselected Picornaviruses which have been altered in one or more capsid proteins compared with wild-type virus, such as Coxsackie virus comprising one or more of the mutations VP3 R96H, VP3 E101A, VP3 A239S, VP2 S164L and VP2 V209. As described in PCT/AU2005/000048 samples of viruses described therein were deposited under the terms of the Budapest Treaty at the Australian Government Analytical Laboratories (National Measurement Institute, 1 Suakin Street (PO Box 385) Pymble NSW 2073 Australia. Isolates CVA21 #272101 (Accession No. NM05/43993), CVA21 #275238 (Accession No. NM05/43991), and CVA21 #272598 (Accession No. NM05/43992), were deposited on 14 Jan. 2005. CVA21-DAFv was deposited on 17 Jan. 2005 under Accession No. NM05/43996.

Methods for the preparation and selection of modified Picornaviruses having the ability to lyse various cancer cells are also described in Newcombe, N. G., et al Enterovirus Capsid Interactions with Decay-Accelerating Factor Mediate Lytic Cell Infection. J. Virol., 2004. 78(3):1431-1439.

Accordingly it will be appreciated that the Picornavirus in the context of the present invention may be a modified Picornavirus, such as described in PCT/AU2005/000048 and in Newcombe et al (2004).

In one embodiment the Picornavirus is a Picornavirus modified in one or more capsid proteins compared with wild-type virus, such as a Coxsackievirus comprising one or more of the mutations VP3 R96H, VP3 E101A, VP3 A239S, VP2 S164L and VP2 V209.

The neoplasm can be a solid neoplasm, such as a sarcoma or carcinoma, or a cancerous growth affecting the hematopoietic system, such as a lymphoid cancer, lymphoma or leukemia. A neoplasm is an abnormal tissue growth, generally forming a distinct mass that grows by cellular proliferation more rapidly than normal tissue growth. Neoplasms show partial or total lack of structural organisation and functional coordination with normal tissue. In the context of this specification a “neoplasm” also referred to as a tumor is intended to encompass hematopoietic neoplasms as well as solid neoplasms. At least some of the cells of the neoplasms may express DAF and or ICAM-1. As demonstrated herein one neoplasm that is particularly suited to the method of the invention is melanoma. Other neoplasms that may be treated by the methods of the invention include breast cancer, brain cancer such as glioblastoma, lung cancer, prostate cancer, colorectal cancer, thyroid cancer, renal cancer, adrenal cancer, liver cancer, leukemia, ovarian cancer, stomach and intestinal cancer etc.

The nucleic acid is typically administered to the mammal in a physiologically acceptable carrier or vehicle, such as physiologically acceptable saline. Administration of the nucleic acid to a mammal indicates that the nucleic acid is administered in such a way that the nucleic acid contacts one or more cells of the neoplasm. The route of administration, as well as the formulation, carrier or vehicle, may depend on the type of neoplasm, its location and the form of the nucleic acid being administered. A wide variety of administration routes may be employed. For example, for an accessible solid neoplasm the route of administration may be direct injection. For a hematopoietic neoplasm the nucleic acid may be administered intravenously or intravascularly. For neoplasms that are not easily accessible in the body, such a metastases and brain tumors, the nucleic acid may be administered through the body of the mammal being treated, such as by intrathecally, intravenously or intramuscularly) so the nucleic acid is transported systemically through the body to the neoplasm. Alternatively the nucleic acid may be administered directly to a single solid neoplasm. The nucleic acid may also be administered subcutaneously, intraperitoneally, topically (such as for treatment of melanoma), orally (such as for treatment of an oral or oesophageal neoplasm), rectally (such as for treatment of a colorectal neoplasm), vaginally (such as for treatment of cervical or vaginal neoplasms), nasally or by inhalation spray (such as for treatment of lung or throat neoplasms).

In general, suitable compositions for administration may be prepared according to methods which are known to those of ordinary skill in the art and accordingly may include a pharmaceutically acceptable carrier, diluent and/or adjuvant.

The compositions comprising the nucleic acid can be administered by standard routes. In general, the compositions may be administered by the parenteral (e.g., intravenous, intraspinal, subcutaneous or intramuscular), oral or topical route. More preferably administration is by the parenteral route.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

It will be understood that treatment of the mammal according to the invention may be undertaken as the sole method of treating a neoplasm in a mammal or may be used in conjunction with other methods for the treatment of neoplasms. Thus, for example, the method may be used with conventional therapy such as chemotherapy and radiotherapy, and where applicable may be used in conjunction with surgical methods. The method may also be used in conjunction with virotherapy, in which live virus is administered to the mammal. Where such combination therapies are undertaken it will be understood that any specific order of the various aspects of treatment may be undertaken, as will be determined by the treating physician. For example, in one treatment regime administration of the nucleic acid may precede surgical means of treatment; which may or may not be followed by chemotherapy and or radiotherapy. Thus, the specific steps of the treatment regime may be undertaken in any order, as determined by the physician.

Treatment of the mammal may comprise a single administration of the nucleic acid or may comprise multiple administrations, such as two, three, four or more administrations. Where the method comprises multiple administrations of the nucleic acid these may be administered at intervals as determined by the physician. Exemplary intervals between multiple administrations are about one day, about two days, about five days, about eight days, about one week, about two weeks, about three weeks, or about one, two or three months. Preferably the administration(s) may be made until the viremic level is about 10⁶ to about 10⁷ TCID₅₀/ml.

The method of the invention may be used in conjunction with immunosuppressant agents. Thus, for example, administration of the nucleic acid according to the invention may precede or follow the establishment of the mammal being treated on immunosuppression therapy, such as with known immunosuppressants for example cyclosporin and variants thereof. In this manner the immune response of the individual being treated will be at least modulated to increase the effectiveness of the transfection and the subsequent lytic action.

In the context of the specification “cell lysis” refers to the disruption of the cell membrane of a cell and the subsequent release of all or part of the content of the cell or the induction of cell death by apoptosis.

The mammal may be any mammal in need of treatment in accordance with the invention, including humans and individuals of any species of social, economic or research importance including but not limited to members of the genus ovine, bovine, equine, porcine, feline, canine, primates and rodents.

In the context of this specification, the language “therapeutically effective amount” is intended to include within its meaning a non-toxic but sufficient amount of a compound or composition of the invention to provide the desired therapeutic effect. The exact therapeutically effective amount of the agent will vary according to factors such as the type of disease of the animal, the age, sex, and weight of the animal, mode of administration. Dosage regima can be adjusted to provide the optimum therapeutic response. For example, several divided doses can be administered daily or the dose can be proportionally reduced as indicated by the exigencies of the therapeutic situation.

In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

### EXAMPLES

The invention will now be described in greater detail by reference to specific examples, which should not be construed as in any way limiting the scope of the invention.

### Materials and Methods

### Cells and Virus

The human melanoma cell line, SK-MeI-28, was obtained from S. J. Ralph (Department of Biochemistry and Molecular Biology, Monash University, Victoria, Australia). Heteroploid human embryonal rhabdomyosarcoma (RD) cells were obtained from Margery Kennett (Entero-Respiratory Laboratory, Fairfield Hospital, Melbourne, Victoria, Australia). Chinese hamster ovary (CHO) cells were obtained from Bruce Loveland (Austin Research Institute, Heidelberg, Victoria, Australia).

Coxsackievirus A21 (CVA21) prototype strain, Kuykendall, was obtained from Dr Margery Kennett (Entero-Respiratory Laboratory) and propagated in SK-MeI-28 cells.

### Antibodies

The anti-ICAM-1 monoclonal antibody (MAb) WEHI, specific for the N-terminal domain of ICAM-1 (Boyd, A. W., et al., Intercellular adhesion molecule 1 (ICAM-1) has a central role in cell-cell contact-mediated immune mechanisms. Proc Natl Acad Sci USA, 1988. 85(9):3095-9) was obtained from Andrew Boyd (Queensland Institute for Medical Research, Queensland, Australia). The anti-DAF MAb IH4, which recognizes the third short consensus repeat (SCR3) of DAF (Coyne, K. E., et al., Mapping of epitopes, glycosylation sites, and complement regulatory domains in human decay accelerating factor. J Immunol, 1992. 149(9):2906-13), was obtained from Bruce Loveland (Austin Research Institute, Heidelberg, Victoria, Australia).

### Cell Culture

SK-MeI-28 and RD cells were maintained in Dulbecco's modified Eagle's medium (DMEM, GIBCO, Invitrogen Corporation, Auckland, NZ) and CHO cells in RPMI 1640 (GIBCO) both supplemented with; 2% v/v Fetal Calf Serum (FCS, GIBCO); 100 μg/ml penicillin/streptomycin (Thermo Trace, Melbourne, Australia) and 2% v/v HEPES Buffer (GIBCO). Cells were grown in monolayer cultures at 37° C. in a 5% carbon dioxide (CO₂) atmosphere.

For cell passage, confluent cell monolayers were washed twice with 10 ml phosphate buffered saline (PBS, Thermo Trace), then incubated for 30 seconds with 10 ml of Trypsin EDTA solution (GIBCO). The trypsin was removed and cells incubated at 37° C. for 1 minute, or until cells lifted from flask surface. Cells were resuspended in approximately 10 ml of growth medium and an appropriate volume of cell suspension was transferred to a new flask (either 75 cm² or 175 cm²) containing 30-50 ml of growth medium. Flasks were then incubated until confluent monolayers formed.

To prepare cell monolayers in 96 well tissue culture plates, 1×10⁴ cells/well were seeded in 100 μl 2% growth medium. 24 well plates used for transfections were seeded with 4×10⁴ cells per well in 500 μl of antibiotic free 2% growth medium. All plates were incubated overnight at 37° C. in a 5% CO₂ atmosphere to achieve 90 to 95% confluency.

### Flow Cytometry

DAF and ICAM-1 surface expression on the cell lines used in this study were analyzed by flow cytometry. Cell monolayers were harvested by incubating with 10 ml versene solution (GIBCO) for 10 minutes at 37° C. and cell suspensions centrifuged at 2000 rpm for 5 mins at 4° C. Dispersed cells (10⁶ cells in PBS) were incubated on ice with the appropriate MAb (5 μg/ml diluted in PBS) for 20 minutes. Cells were washed with PBS and pelleted at 1000×g for 5 minutes, resuspended in 100 μl of 1:100 dilution of R-phycoerythrin-conjugated F(ab′)₂ fragment of goat anti-mouse immunoglobulin (DAKO A/S, Copenhagen, Denmark) and incubated for 20 minutes on ice. Cells were washed, pelleted and resuspended in PBS, and expression levels of ICAM-1 and DAF were acquired using a FACStar Analyser (Becton Dickenson, Sydney, Australia). Data was analyzed using Weasel version 2.1 software (www.biotechcentre.net.au/cytometry).

### Preparation of Coxsackievirus A21 Viral RNA

Growing and Isolating Virus

Viral stocks of CVA21 were grown in confluent monolayers of SK-MeI-28 cells cultured in 175 cm² tissue culture flasks. Infected cells were incubated for 24 hours at 37° C. or until 100% viral cytopathic effect (CPE) was observed. Flasks were frozen at −80° C. for 1 hour then thawed at 37° C. The viral suspension was transferred to a 50 ml Falcon tube, vortexed for 30 seconds then centrifuged at 2000 rpm for 5 minutes at 4° C. to remove cell debris. Supernatant was transferred to Beckman ultracentrifuge tubes and spun at 36 000 rpm for 2.5 hours at 4° C. in a Beckman XL-90 ultracentrifuge (SW41Ti Rotor). Virus pellet was resuspended in 200 μl of supernatant and transferred to a 1.5 ml microfuge tube for RNA extraction.

Extraction of Viral RNA

Trizol® LS reagent (GIBCO BRL, Life Technologies Grand Island, N.Y., USA) and chloroform were added to the resuspended virus and incubated at room temperature (RT) for 3 minutes. The mixture was spun at no more than 12000×g for 15 minutes at 4° C. and RT isopropyl alcohol was added to the upper aqueous phase, incubated for 10 minutes at RT. The RNA was pelleted at no more than 12000×g for 10 minutes at 4° C. and washed with 75% ethanol, centrifuged at 2500 rpm for 5 minutes at 4° C. (Eppendorf Centrifuge 5417R, Hamburg, Germany), air-dried, resuspended in RNase free distilled water (dH₂O) and stored at −80° C.

Estimation of Viral RNA Concentration

The concentration of viral RNA (vRNA) in the extracted sample was estimated using ultra-violet (UV) spectrophotometry. The sample was diluted in RNase free distilled water and absorbance read at 260 nm (A₂₆₀) using Bio-Spec-mini (SHIMADZU Corporation, Japan). RNA concentration was calculated using the following equation:

RNA Concentration (μg/ml)=A₂₆₀×40×Dilution Factor (Ausubel et al., ibid)

Denaturing Agarose Gel Electrophoresis

The presence of vRNA was visualized using denaturing agarose gel electrophoresis. RNA samples (5 μl and 10 μl) and a 0.3-6.9 kb RNA marker (Roche Diagnostics, Indianapolis, Ind. USA) were denatured by heating at 65° C. for 5 minutes in 15 μl of denaturant (22.5% v/v 12.3 M Formaldehyde, 0.1% v/v ethidium bromide (EtBr, 10 mg/ml stock), 64.4% Formamide, 13% 10×MOPS buffer). Samples were separated on a 1% agarose gel containing 10.4% v/v 10×MOPS buffer, 18.8% v/v 12.3 M Formaldehyde, in 1×MOPS buffer at 100 volts for 1-1.5 hours. The gel was photographed under UV light then washed in RNase free dH₂O for 10 minutes before being further processed for northern blot analysis (see below).

Northern Blot and CVA21 3′ RNA Probe Hybridization

RNA bands on the agarose gel were transferred onto a nylon membrane via northern blot capillary transfer. The gel was washed three times (10 minutes in RNase free dH₂O, 15 minutes in 0.05 M sodium hydroxide (NaOH), 10 minutes in 10×SSC buffer) then transferred onto the nylon membrane (Zeta-Probe® BIORAD Laboratories, Hercules, Calif., USA) overnight (approx 16 hours) in 20×SSC buffer. The membrane was soaked in 10×SSC for 10 minutes, air-dried and baked at 80° C. for 2 hours to fix the RNA.

The fixed membrane was probed for CVA21 RNA using a DIG-11-dUTP (Digoxigenin-11-2′-deoxy-uridine-5′-triphosphate) labeled DNA Probe specific for a 420 nucleotide, 3′ region of CVA21. The probe was a kind gift from Miss Erin Haley (Department of Immunology and Microbiology, University of Newcastle, Australia) and was prepared using DIG-dUTP PCR incorporation. The membrane was incubated in a sealed plastic bag containing 10 to 15 ml Hybridization Buffer (0.25 M Na₂HPO₄, pH 7.2, 1 mM EDTA, 20% SDS, 0.5× Block (Blocking Reagent (Roche, Indianapolis, USA) diluted in Maleic Acid Buffer; 100 mM Maleic Acid, 150 mM NaCl pH 7.5) in a 68° C. water bath for 1 hour whilst agitating. The probe was denatured at 99° C. for 10 minutes and placed on ice for 2 minutes before 4 μl was added to the bag with fresh Hybridization Buffer, the bag resealed and incubated at 68° C. overnight (approximately 16 hours). The membrane was washed twice with pre-warmed (65° C.) Wash Buffer (20 mM Na₂HPO₄, 1 mM EDTA, 1% SDS) for 20 minutes at 65° C., then rinsed with Buffer A (0.1 mM maleic acid, 0.3% Tween 20, pH 8.0, 3M NaCl) for 5 minutes at RT. Buffer A was removed and the membrane blocked with 1× Blocking Buffer (Buffer A with 0.5× Block) for 1 hour at RT whilst agitating. The probe was detected by incubating in 15 ml of a 1/5000 dilution of Anti-DIG-Alkaline-Phosphatase in 1× Blocking Buffer for 30 minutes and unbound antibody removed with four 10 minute washes in Buffer A at RT. The membrane was equilibrated with Substrate Buffer (0.1 M Tris, 0.1 M NaCl, 50 mM MgCl₂, pH 9.5) for 5 minutes at RT, then incubated in 15 ml CSPD substrate (1:100 dilution of CSPD (Disodium 3-(4-methoxyspiro {1,2-dioxetane-3,2′-(5-chloro)tricyclo[3.3.1.1^{3,7}]decan}-4-yl)phenyl phosphate), Roche in Substrate Buffer) for 5 minutes at RT and then air dried. CSPD is a chemiluminescent substrate for alkaline phosphatase which, when dephosphorylated, emits light detectable on X-ray film. The membrane was exposed to Hyperfilm™ (Amersham Pharmacia Biotech UK, Buckinghamshire, England) for 30 minutes and developed using a DuPont QC1-RT processor (Sterling Diagnostic Imaging, Hertfordshire England).

### In Vitro Liposome-Mediated CVA21 Viral RNA Transfection

Transfection Optimization

Several concentrations of Lipofectamine 2000 (Invitrogen Life Technologies, Carlsbad Calif., USA) ranging from 0.5 to 10 μl per well were tested for cytotoxicity on SK-MeI-28, CHO and RD cells by incubating the cells in medium containing Lipofectamine 2000 for up to 48 hours. An optimal concentration of Lipofectamine 2000 suitable for the three cell lines (2 μl per well) was then used for all subsequent transfections.

SK-MeI-28, CHO and RD Cell Transfections

Sk-MeI-28, CHO and RD cell transfections were carried out in 24 well tissue culture plates seeded as described herein.

Preparation of Viral RNA:Lipofectamine 2000 Complex

For each transfection sample, the vRNA:Lipofectamine 2000 complexes (vRNA:lipid) were prepared according to manufacturer's instructions. Briefly, various amounts of vRNA and 2 μl Lipofectamine 2000 were diluted separately in 50 μl serum and antibiotic free media and gently mixed. Five minutes following Lipofectamine 2000 dilution, the corresponding vRNA and lipid solutions were combined and gently mixed. To allow vRNA:lipid complexes to form, the mixture was incubated at RT for 45 minutes.

One hundred microliters of transfection complex was added to each well of the prepared 24 well plates containing cells and medium and mixed by gentle rocking. Control wells contained vRNA only, lipid only and CVA21 live virus and were also diluted to 100 μl in serum and antibiotic free media. The plates were incubated at 3 7° C. in 5% CO₂ atmosphere. After 6 hours, 600 μl 2% FCS DMEM without antibiotics was added to each well and incubated for up to 48 hours.

Evaluation of Cytopathic Effect

A qualitative measure of virus production was determined by microscopic evaluation of viral cytopathic effect (CPE) at various time-points. Each well was inspected for characteristic cell rounding, nuclear condensation, and cell detachment from plate surface. CPE was recorded on a percentage scale from 0, 25, 50, 75 and 100%.

Collection and Storage of Transfection Supernatants

At 0, 12, 24, and 48 hours post transfection, the supernatant and cells from each well were collected for viral titre analysis. Any attached cells were scraped from the plate surface using an upturned pipette tip and the entire contents of the well transferred to a 1.5 ml microfuge tube. The cells and supernatant were vortexed for 30 seconds, frozen at −80° C. and thawed to release any intracellular virus particles. Cellular debris was pelleted at 2000 rpm for 5 minutes at 4° C. (Eppendorf Centrifuge 5417R) and supernatant stored at −80° C.

Passage of CHO and RD Cell Supernatants

Each supernatant sample from the CHO and RD cell transfections was passaged onto fresh plates of either CHO or RD cells respectively. Plates were prepared as previously described, and 200 μl of each transfection supernatant was added to the appropriate wells. The cells were monitored for CPE for 48 hours.

Virus Infectivity Assays

Confluent monolayers of SK-MeI-28 cells in 96 well tissue culture plates were infected in triplicate with 100 μl of 10-fold serial dilutions of transfection supernatant and incubated at 37° C. in 5% CO₂ atmosphere for 72 hours. CPE in each well was microscopically evaluated as described herein, then fixed and stained with a crystal violet solution (0.1% crystal violet, 20% methanol, 20% Formalin, 60% PBS). A well was scored positive (+) if any CPE was present regardless of the percentage observed. Viral titres expressed as 50% tissue culture infectious doses (TCID₅₀/ml) were calculated using the following equation:

Log TCID₅₀=10^{1+d(S-0.5)} [22]

Where; d=log of the dilution (=1 for 10 fold dilution);
S=sum of ratios of CPE positive:CPE negative wells per triplicate row
   (Dougherty, R. M., Animal virus titration techniques, in Techniques in experimental virology., R. J. C. Harris, Editor. 1964, Academic Press,: New York. pp. 169-223).

### In Vivo Established Subcutaneous Human Melanoma Xenograft Model

Experimental Animals

Female NOD-SCID mice, 4 to 6 weeks of age, were obtained from the University of Newcastle Animal Facility and housed in pathogen free conditions within the animal handling facility of the University. Animals were housed in cages of four or five mice, in a controlled room cycling 12 hours of light and 12 hours of darkness and were given food and water ad libitum. All animal work was performed under guidelines approved by The University of Newcastle Animal Care and Ethics Committee.

Cell Preparation and Injection Protocol

SK-MeI-28 cells were grown in 10% FCS DMEM, harvested with trypsin and washed once in 2% FCS DMEM and twice in sterile PBS. Cell viability was evaluated by trypan blue staining, only cell fractions containing >95% viable cells were used for injection. Finally, cells were resuspended in sterile PBS to a concentration of 3×10⁷ cells/ml.

Animals were anaesthetized with 100-150 μl intra-peritoneal (i.p.) injections of a 1/10 dilution of Rompun (Bayer, NSW, Australia) and Ketamine (Parnell Laboratory, NSW, Australia) in PBS prior to injection. SK-MeI-28 cells (3×10⁶ in 100 μl PBS) were injected subcutaneously, between the shoulder blades of each mouse. This site was chosen both to minimize discomfort to the mouse and prevent the mouse from interfering with the tumour.

Estimation of Tumour Volume

Tumour volumes were monitored daily and periodic measurements were made using electronic digital calipers (Dick Smith Electronics, Australia) measuring in millimeter increments to one decimal place. Before measurement, the tumour area was saturated with 75% ethanol to eliminate error associated with thickness of the fur. Two intersecting measurements (length and width) were made of each tumour and volume (V) was calculated using the following equation for the volume of a spheroid:

V(mm³)=π/6a.b²
Where; a=length (mm), b=width (mm), a>b.

Standard error (SE) of tumour volumes for each group were calculated using:

SE=SD/√n

### In Vivo Intratumoural Administration of CVA21 Viral RNA

One day prior to treatment injection, all tumour volumes were measured and the mice randomized into 4 groups based on tumour size. Groups of mice received 1 of 4 treatments; vRNA:lipid complex (n=5), vRNA only (n=4), lipid only (n=4) or CVA21 live virus (n=4).

Treatment Preparation and Injection Protocol

Viral RNA:Lipofectamine 2000 complexes were prepared as described herein, maintaining the optimum determined vRNA:lipid (μg:μl) ratio. 50 μl transfection complexes containing 2 μg vRNA and 4 μl Lipofectamine 2000 were prepared along with control treatments of 50 μl dilutions of 2 μg vRNA only and 4 μl Lipofectamine 2000, all in serum and antibiotic free DMEM. The three treatments were incubated at RT for 45 minutes prior to injection. In addition, 50 μl doses of stock CVA21 live virus (10⁹ TCID₅₀/ml) were kept on ice prior to injection.

Animals were anesthetized by inhalation using 4% isofluorane (ISOFLO™, Abbott Australasia, NSW Australia) for treatment injection. Each treatment was administered via a single intra-tumoural (i.t.) 50 μl injection with a 30.5 gauge insulin syringe.

All four treatments were administered on day 0, and vRNA:lipid complex, vRNA only and lipid only treatments were injected a second time on day 8.

Sera Collection Via Saphenous Vein Bleed

A blood sample (approx 75 μl) was collected at various time-points via saphenous vein bleed. Blood was collected using non-heparinised capillary tubes (Hirschmann Laborgerate, Germany) and transferred to 0.5 ml microfuge tubes. Samples were allowed to clot at 4° C. for 10-20 minutes and sera separated by centrifugation at 12 000 rpm for 5 minutes 4° C. (Eppendorf Centrifuge 5417R). Serum samples of between 10 to 40 μl were collected and stored at −80° C.

Sacrifice Via Isofluorane Overdose

All mice were sacrificed using an inhalation overdose of 4% isofluorane (Abbott Australasia). A final blood sample was collected via heart puncture and sera isolated and stored at −80° C.

Viral RNA Extraction from Sera

Viral RNA from infectious virions present within serum samples of mice were extracted using a QIAamp® Viral RNA Mini Kit Mini-spin Protocol (QIAGEN Pty Ltd, Victoria, Australia). Briefly, 10 μl sera was added to 60 μl RNase free dH₂O and 280 μl AVL Buffer containing ‘carrier RNA’ (supplied), the mixture vortexed and incubated at RT for 10 minutes to lyse intact virus and inactivate RNases. 280 μl of ethanol (98-100%) was added, mixed by vortexing and the entire volume loaded onto a QIAamp® Mini Spin column silica-gel membrane, and then centrifuged at 8000 rpm for 1 minute at 22° C. (Eppendorf Centrifuge 5417R). The membrane was washed twice with 500 μl of supplied washing buffers (AW1 and AW2) centrifuging at 8000 rpm 1 minute, between each wash to remove any contaminants. Pure RNA was eluted in 40 μl of AVE elution buffer by spinning at 8000 rpm for 1 minute, collected and stored at −80° C.

To generate a set of standards for quantitative real-time-PCR, series of standard vRNA samples were extracted from known concentrations of CVA21 in the same manner as above. A ten-fold serial dilution of stock CVA21 of known titre was prepared in RNase free dH₂O, viral RNA was extracted from samples within this series (10¹-10⁷ TCID₅₀/ml) and stored in aliquots at −80° C. For each vRNA extraction, a negative control dH₂O sample was also extracted.

Quantitation of Sera Viral Load Via Real-Time RT-PCR

Viral load (TCID₅₀/ml) within a sera sample was quantified using real-time reverse-transcriptase polymerase chain reaction (qRT-PCR). A dual-labeled fluorogenic probe specific for a sequence within the VP3 region of the CVA21 Kuykendall Strain (KKVP3, Applied Biosystems, CA, USA) containing a 5′-reporter dye 6-carboxyfluorcein (FAM) and a 3′-quencher dye 6-carboxytetramethylrhodamine (TAMRA) was used to quantify the amount of vRNA within a sample. Sera and standard RNA samples were analyzed using Platinum® Quantitative RT-PCR ThermoScript™ One-Step System (Invitrogen Life Technologies, Carlsbad Calif., USA). The sequences of the KKVP3 probe and the forward and reverse primers (both primers from GeneWorks Pty Ltd, Hindmarsh, SA, Australia) are shown in Table 2.

TABLE 2

Primer and Probe sequences used for Real-time
PCR of CVA21 Kuykendall Strain VP3 region.

| Primer or | |
|---|---|
| Probe | Sequence |
| Forward | 5′ GAG CTA AAC CAC CAA CCA ATC G 3′ |
| Primer | |
| Reverse | 5′ CGG TGC AAC CAT GGA ACA A 3′ |
| Primer | |
| KKVP3 | 5′ CAC ACA CAT CAT CTG GGA 3′ |
| Probe | |

Each 25 μl reaction contained 5 μl eluted vRNA, 2 μl RNase free dH₂O, 12.5 μl 2× ThermoScript Reaction Mix (containing 0.4 mM of each dNTP, 6 mM MgSO₄), 0.5 μl ROX Reference Dye, 0.5 μl ThermoScript Plus/Platinum® Taq Mix (containing a mixture of reverse transcriptase and Taq DNA polymerase), 1 μl of each 10 μM primer (Forward and Reverse) and 2.5 μl KKVP3 Probe (2.5 μM). The negative control water extractions were also analyzed along with an RNase free dH₂O, non-template control during each run. PCR samples were incubated for 30 minutes at 60° C. for cDNA synthesis and 5 minutes at 95° C. to deactivate the reverse transcriptase, denature the RNA/cDNA hybrid, and activate the Platinum® Taq DNA Polymerase. Samples were then cycled 40 times through 15 seconds at 95° C. (denaturation) and 1 minute 60° C. (annealing and extension) using the ABI PRISM 7000 Sequence Detection System (Applied Biosystems, Victoria, Australia). Fluorescence data was collected during the annealing-extension step, and analyzed using ABI Prism SDS software Version 1.1 (Applied Biosystems). The reporter dye (FAM) signal was measured relative to the reference dye (ROX) to normalize well-to-well non-PCR related fluctuations. The mean baseline emission levels were calculated between cycles 6 and 15 and the threshold level was set within the linear region of exponential amplification.

Virus Infectivity Assays of Sera

The infectivity of virus present within the mouse serum was evaluated by titration on 96-well plates of SK-MeI-28 cells with a 10-fold dilution series of serum as “Virus infectivity assays” as above. In this case however 4.5 μl of sera was used to start each dilution series at 10⁻². Fifty percent tissue culture infectious doses were calculated as previously described (see “Virus infectivity assays” as above).

### Tumour Analysis

Macroscopic tumour characteristics were evaluated following sacrifice of each animal. The far and skin covering the back of each mouse was removed and photographs of the remaining tumour mass were taken. Tumour sections were isolated for further analysis and washed in PBS three times. Tumour sections were weighed and placed in Lysing matrix D tubes containing ceramic homogenization beads (BIO 101 Systems, Qbiogene, CA, USA), 600 μl PBS was added to each sample and held on ice. Samples were homogenized for 45 seconds (FastPrep™ FP120 BIO 101 ThermoSavant, Integrated Sciences, Willoughby, NSW, Australia) and returned to ice for 5 minutes. Cell debris was pelleted at 8000 rpm for 10 minutes at 4° C. (Eppendorf Centrifuge 5417R) and supernatant collected and stored at −80° C. Virus infectivity assays of tumour lysate supernatants were carried out as described in “Virus infectivity assays” as above.

### Statistical Analysis

One-way analysis of variance (ANOVA) followed by a Newman-Keul's post-test was used to examine the differences between tumour volumes of each different treatment group. The significance level was set at P<0.05 for all tests. GraphPad Prism version 4 for Macintosh statistical software (GraphPad Software, San Diego, Calif., USA) was used for all statistical tests.

### Example 1

### Cell Surface Expression of ICAM-1 and DAF on SK-MeI-28, RD and CHO Cells

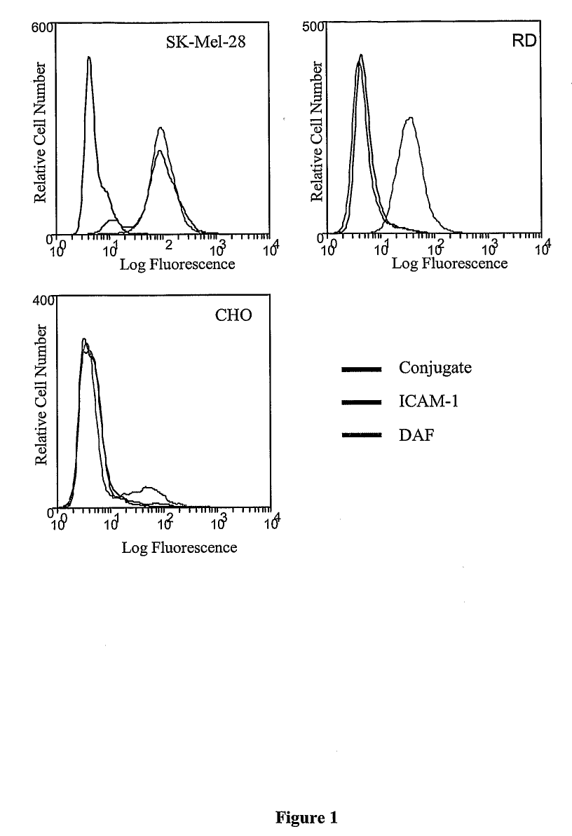

The expression of the Coxsackievirus A21 cellular receptors, intercellular adhesion molecule-1 (ICAM-1) and decay-accelerating factor (DAF) on the surface of cells used in this study were analyzed by flow cytometry. Each cell line was incubated with either an anti-ICAM-1 MAb (WEHI) or anti-DAF MAb (IH4) prior to incubation with a fluorescent conjugate and subsequent laser scanning. ICAM-1 expression was detected at high levels on the human melanoma cell line SK-MeI-28, while neither the human embryonal rhabdomyosarcoma (RD) cells nor chinese hamster ovary (CHO) cells expressed ICAM-1. High levels of surface DAF expression were limited to the SK-MeI-28 and RD cells (FIG. 1).

### Example 2

### Characterization of Viral RNA Extracted from CVA21 Virions

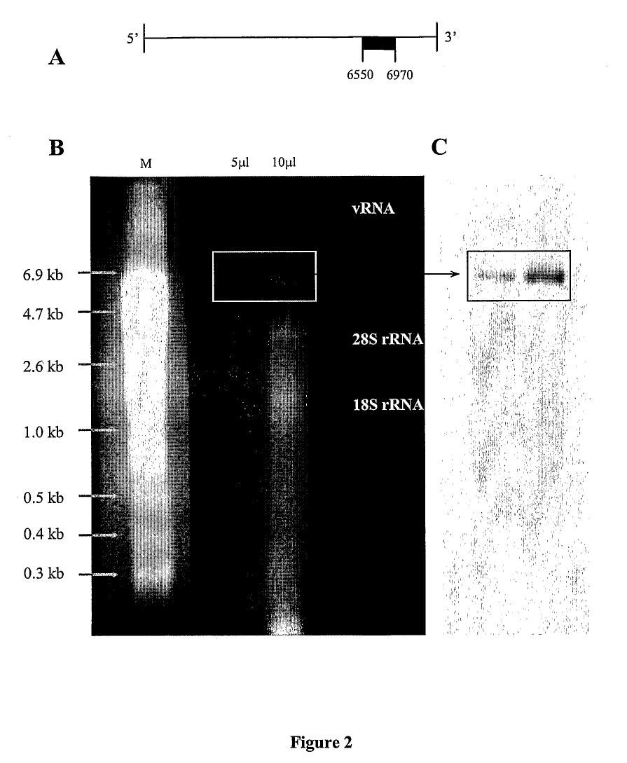

The presence of intact full-length CVA21 vRNA following Trizol® extraction was assessed using denaturing agarose gel electrophoresis. Under ultra-violet light examination, three distinct RNA bands were visualized on the gel, two ribosomal RNA (rRNA) bands (28S rRNA at ˜4.2 kb, 18 S rRNA at 2.2 kb), and a viral RNA band at ˜7 kb. (FIG. 2B). The RNA band was transferred to nylon by northern blot capillary transfer and hybridized with a DIG-1′-UTP labeled DNA probe specific for a 420 nucleotide 3′ region of CVA21 RNA (FIG. 2A). An intense band of CVA21 RNA was visualized following hybridization and chemi-luminescent detection (FIG. 2C). The visualized band corresponded to the vRNA band observed on the agarose gel and confirmed the presence of full-length CVA21 RNA.

### Example 3

### In Vitro CVA21 Viral RNA Transfection

### 3.1. Transfection Optimization

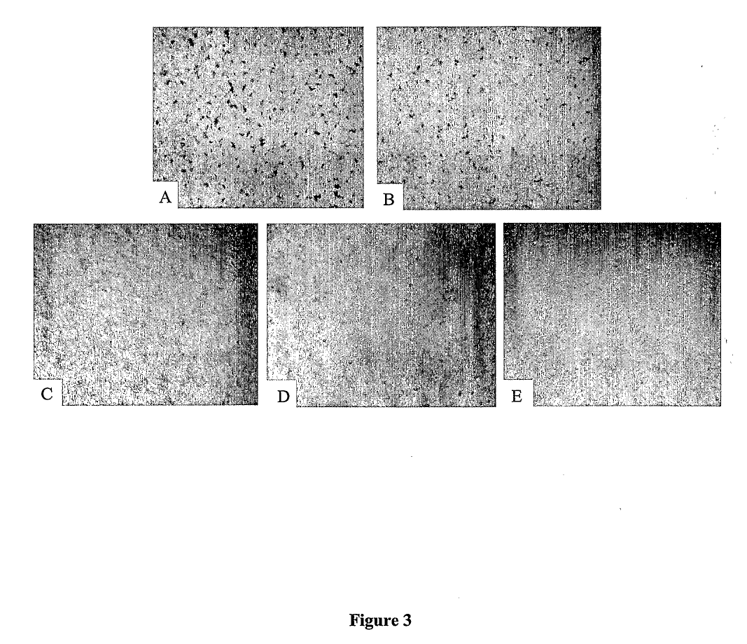

Various concentrations of Lipofectamine 2000, a cationic lipid used for cell transfection, were tested for levels of cell toxicity on SK-MeI-28, RD and CHO cells. Lipid volumes ranging from 0.5 μl to 10 μl diluted in 100 μl of growth medium, were added to monolayer cultures of cells (4×10⁴ cells/well) in 24-well plates. Following incubation for 24 hours at 37° C. in 5% CO₂, cell monolayers were microscopically examined for signs of cell toxicity. Despite manufacturer's recommendations, high concentrations of lipid (5 to 10 μl/well) were cytotoxic and induced almost complete cell death within 24 hours in SK-MeI 28 cells (FIGS. 3 A and B), RD and CHO cells (data not shown), whereas lower lipid concentrations (0.5 to 3 μl/well) were not toxic to SK-MeI 28. Two microlitres per well was found to be the highest concentration of Lipofectamine 2000™ tolerated by all three cell lines, SK-MeI-28 (FIG. 3C), RD (FIG. 3D), CHO cells (FIG. 3E) and was used for all subsequent transfections to maximize RNA delivery.

3.2. Transfection of SK-MeI-28 Cells with CVA21 Viral RNA

3.2.1 Examination of Cytopathic Effect Development in Monolayer Cell Cultures of SK-MeI-28 Cells

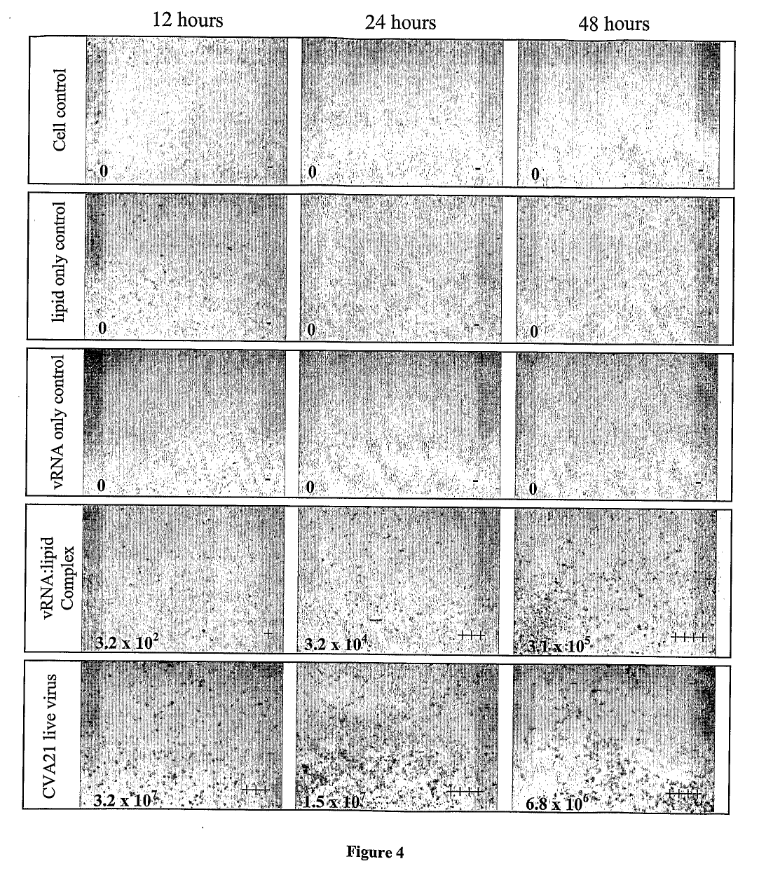

The progression of CVA21 induced CPE was microscopically monitored following transfection of SK-MeI-28 cells with 1 μg CVA21 RNA (vRNA) complexed with 2 μl Lipofectamine 2000™ (vRNA:lipid). Twelve hours post infection (PI), cell rounding, nuclear condensation and cell lysis characteristic of enteroviral lytic infection [23], was observed in both the CVA21 live virus control wells and the vRNA:lipid complex treated wells. No signs of viral infection were observed in the vRNA only or lipid only treated control wells. Cell monolayers treated with live intact CVA21 virions (1.6×10⁴ TCID₅₀/well) exhibited complete CPE at 24 hours PI, while vRNA:lipid complex treated monolayers required 24 to 48 hours incubation before total cell lysis was observed (FIG. 4). Despite the longer time required for complete CPE, the vRNA:lipid treated cells exhibited identical morphology of cell death as the virally infected cells.

3.2.2. Level of CVA21 Progeny Virus Production Following Transfection of SK-MeI-28 Cell Monolayer Cultures with CVA21 vRNA

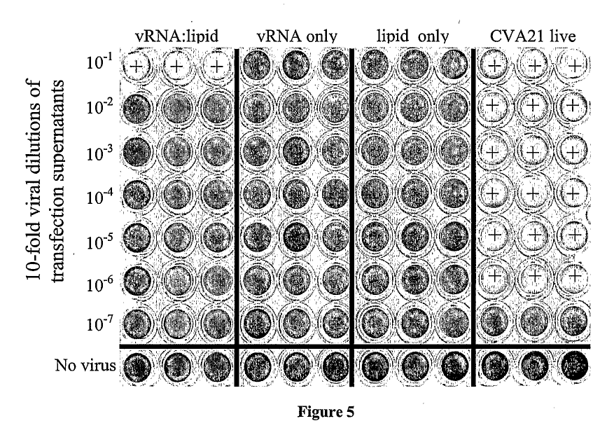

The levels of infectious CVA21 production in SK-MeI-28 cell monolayer cultures following CVA21 vRNA transfection were determined by lytic cell infectivity assays. SK-MeI-28 cell monolayers in 96-well plates were infected with 10-fold serial dilutions of cell supernatants harvested at 12, 24 and 48 hours post-transfection. CPE was microscopically evaluated after 72 hours incubation, when monolayers were fixed and stained with a crystal violet solution (FIG. 5). The fifty percent infectious titres (TCID₅₀/ml) for each supernatant sample were calculated using the method of Reed and Muench [61] as described in section 2.6.3. No infectious virus was detected in initial inoculum of lipid only, vRNA without lipid or cell control preparations. In contrast, at 12 hours post-transection infectious virus was observed in both the vRNA:lipid complex (3.2×10² TCID₅₀/ml) and CVA21 live virus (from 1.6×10⁴ initial inoculum to 3.2×10⁷ TCID₅₀/ml) treated cell supernatants. At 24 hours post transfection, the titre of the vRNA:lipid complex treated supernatant increased to 3.2×10⁴ TCID₅₀/ml and 3.1×10⁵ TCID₅₀/ml after 48 hours. No increases in virus levels in CVA21 live virus treated supernatants were detected between 12 and 24 hours post-transfection, as complete CPE occurred between these time-points. As a consequence of the lack of viable cell substrate, at 48 hours post infection, the live virus treated supernatant had fallen in titre to 6.8×10⁶ TCID₅₀/ml (FIG. 4). No infectious virus was detected in the supernatants of the cell control, or vRNA treated cells at any time throughout the examination period (0-48 hours).

3.3. Transfection of RD and CHO Cells with CVA21 Viral RNA

RD and CHO cells were transfected with CVA21 vRNA and monitored for CPE and production of infectious CVA21 progeny virus, to assess the ability of CVA21 vRNA to replicate in cells lacking the CVA21 cellular receptors ICAM-1 and DAF. In addition, the development of a lytic infection within these normally resistant cells would confirm that the vRNA:lipid preparation did not contain infectious intact CVA21 virions, and that indeed the replication of infectious vRNA was the source of progeny virus observed in SK-MeI-28 transfections.

3.3.1. Examination of Cytopathic Effect Development in Monolayer Cell Cultures of RD and CHO Cells

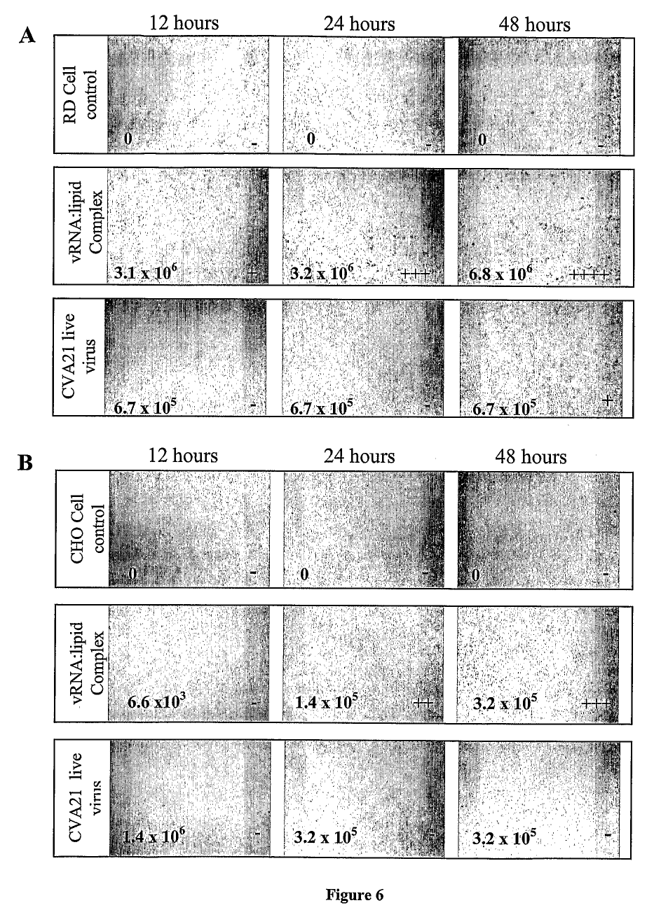

The progression of CVA21 induced CPE was microscopically monitored in monolayer cell cultures of RD and CHO cells (4×10⁴/well) in 24-well plates following lipid-mediated transfection with CVA21 vRNA. RD cells exhibited characteristics of enteroviral lytic infection, including cell rounding and cell lysis, 12 hours post-transfection, with complete lysis observed after 48 hours incubation (FIG. 6A). The development of CPE in CHO cells was not observed until 24 hours post-transfection and increased to 75% of the cells displaying CPE following 48 hours incubation (FIG. 3.6B). RD and CHO cell monolayers treated with an initial dose of 3×10⁴ TCID₅₀/well of intact CVA21 virions did not display any significant CPE after 48 hours. These results are consistent with the finding that neither of these cell lines express ICAM-1 or DAF, the cell surface receptors required for natural lytic CVA21 infection (see herein above).

3.3.2. Level of CVA21 Progeny Virus Production in RD and CHO Cell Monolayer Cultures Following Transfection of CVA21 vRNA

The levels of infectious CVA21 production in RD and CHO cell monolayer cultures following CVA21 vRNA transfection were determined by lytic cell infectivity assays of transfection supernatants, on SK-MeI-28 cell monolayers in 96-well plates (see herein above). Fifty percent infectious titres (TCID₅O/ml) of progeny virus increased in both RD and CHO cell monolayer cultures following transfection with CVA21 vRNA:lipid complex (FIG. 6). No increase in viral titre was observed in the RD or CHO cells inoculated with intact CVA21 virions, with only residual inoculum virus detected in the supernatants.

3.3.3. Examination of Cytopathic Effect Development in RD and CHO Cell Monolayer Cell Cultures Following Passage of Transfection Supernatants Containing Infectious CVA21

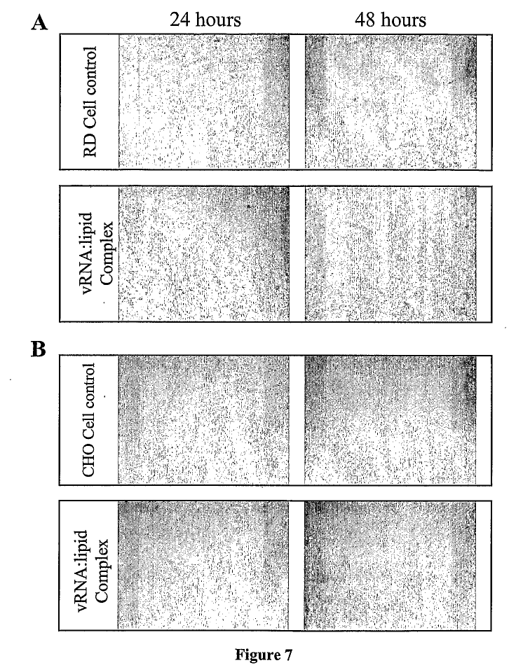

Cell supernatants containing CVA21 produced from the transfection of RD and CHO cells with CVA21 vRNA were passaged onto fresh monolayer cell cultures of RD and CHO cells to assess the selective infectivity of this progeny CVA21. RD and CHO cells are normally unsusceptible to CVA21 lytic infection due to the lack expression of surface ICAM-1 (See FIG. 1). No CPE was observed microscopically in either the RD or CHO cells 48 hours PI with cell supernatants (FIG. 7).

### Example 4

### In Vivo Production of Infectious CVA21 Via Intra-Tumoural Administration of CVA21 vRNA:Lipid Complex

Four groups of NOD-SCID mice bearing established subcutaneous SK-MeI-28 human melanoma xenografts were injected intra-tumourally with one of four treatments; (i) CVA21 vRNA:lipid complex (2 μg RNA; 4 μl lipid), (ii) 2 μg vRNA only, (iii) 4 μl lipid only, (iv) CVA21 live virus (5×10⁶ TCID₅₀ per site) all diluted to 50 μl in sterile DMEM. Initial treatments were administered on day 0, and a subsequent treatment of vRNAlipid complex, vRNA only and lipid only were administered to the appropriate groups on day 8.

4.1. Quantitation of CVA21 Viral Load by Real-Time RT-PCR

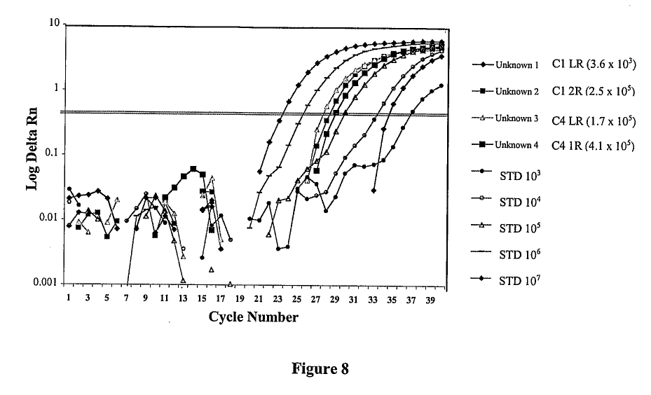

Blood samples were collected at various time-points. Viral RNA was extracted from serum and screened for CVA21 vRNA by real-time RT-PCR. To generate a standard curve, a dilution series of vRNA extracted from a CVA21 stock preparation were amplified in parallel. The threshold level was set within the linear region of exponential amplification and only samples reaching this threshold were deemed positive for CVA21 vRNA (FIG. 8). Viral titres were calculated for the unknown samples by comparing the cycle at which a sample reached threshold (threshold cycle or CT value) to the CT of the standard samples extracted from a stock preparation of CVA21 of known titre.

CVA21 vRNA was only detected in the mice injected with live CVA21 in blood samples collected 2 hours (Day 0), 2 days and 5 days post-injection. A second treatment of vRNA:lipid, lipid only or vRNA only was administered to the appropriate mice on day 8. On day 16 post-injection, CVA21 vRNA was detected in the serum samples of 4/5 mice in the vRNA:lipid complex group (average 2.6×10⁵ TCID₅₀/ml), 2/2 mice in the CVA21 live virus group (average 3.5×10⁶ TCID₅₀/ml) and 2/2 vRNA only treated mice (average 2.6×10⁵ TCID₅₀/ml) tested. On subsequent screening days (days 23, 30, 37 and 44), the same 4 mice in the vRNA:lipid complex group tested positive for CVA21 vRNA, with average levels for the group increasing from 5.6×10⁵ TCID₅₀/ml on day 23 to 1.6×10⁶ TCID₅₀/ml on Day 44. One mouse from the vRNA:lipid complex group never tested positive for CVA21 vRNA during the entire testing period. Levels of CVA21 vRNA detected in the sera of mice treated with CVA21 vRNA only also increased from day 23 (4.1×10⁵ TCID₅₀/ml) to day 44 (1.3×10⁶ TCID₅₀/ml). All mice from the CVA21 live virus treated group tested positive for CVA21 vRNA for every day they were tested, with average levels of 2.0×10⁷ TCID₅₀/ml on day 37. All mice treated only with lipid tested negative for CVA21 vRNA for the duration of the experiment (Table 3).

Days Post Injection

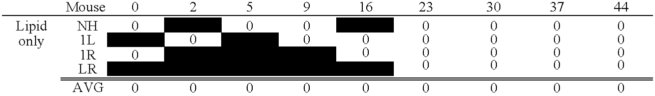

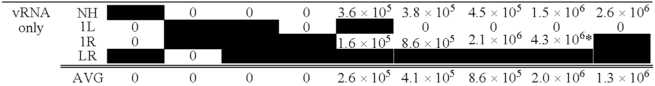

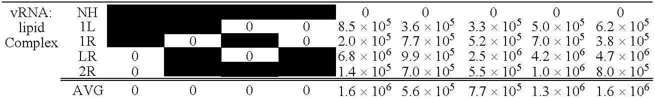

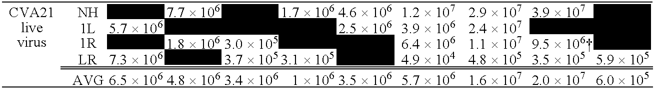

### 4.2 Quantitation of CVA21 Viral Load by Lytic Cell Infectivity Assays

To assess if positive real-time RT-PCR results correlated to the presence of infectious CVA21 in the serum, lytic cell infectivity assays of post mortem blood samples were performed. SK-MeI-28 cell monolayers in 96-well plates were inoculated with 10-fold dilutions of sera. Following incubation for 72 hours at 37° C. in 5% CO₂ the wells were microscopically examined for CPE, fixed and stained with a crystal violet solution (see herein above). All serum samples that tested positive for CVA21 vRNA by real-time RT-PCR, including mice treated with only vRNA, were found to contain infectious CVA21. The infectious titres of the samples were similar to those determined by real-time RT-PCR (Table 4).

TABLE 4
Infectious CVA21 Serum Viral Load of Mice Bearing Subcutaneous
Melanoma Xenografts Treated with Intra-tumoural CVA21 vRNA: lipid
Complex Determined by Lytic Cell Infectivity Assay on SK-Mel-28 Cells.

| | | Serum Viral | |
|---|---|---|---|
| | | Load | Average |
| | Mouse | (TCID₅₀/ml) | (TCID50/ml) |
| lipid only | NH | 0 | 0 |
| | 1L | 0 | |
| | 1R | 0 | |
| | LR | 0 | |
| vRNA only | NH | 1.8 × 10⁶ | 8.4 × 10⁵ |
| | 1L | 0 | |
| | 1R | 5.6 × 10⁵ | |
| | LR | 1 × 10⁶ | |
| vRNA: lipid | NH | 0 | 1.6 × 10⁶ |
| Complex | 1L | 1.8 × 10⁵ | |
| | 1R | 3.2 × 10⁵ | |
| | LR | 5.6 × 10⁶ | |
| | 2R | 1.8 × 10⁶ | |
| CVA21 live | NH | 1.0 × 10⁷ | 1.2 × 10⁷ |
| virus | 1L | 3.2 × 10⁷ | |
| | 1R | 3.2 × 10⁶ | |
| | LR | 6.8 × 10⁵ | |

Post mortem serum samples from four groups of mice 44 days following treatment with CVA21 vRNA: lipid complex, vRNA only, lipid only or CVA21 live virus were tested for the presence of infectious CVA21 by lytic cell assay. Cell monolayers were inoculated with 10-fold dilutions of serum and incubated at 37° C. for 72 hours before being microscopically examined for CPE and stained with a crystal violet solution. End point infectious titres (TCID₅₀/ml) were calculated using the method of Reed and Muench ibid.

### Example 5

### The Oncolytic Action of CVA21 in Human Melanoma Xenografts

5.1. Reduction in Tumour Volume of Melanoma Xenografts in NOD-SCID Mice by CVA21-Mediated Oncolysis

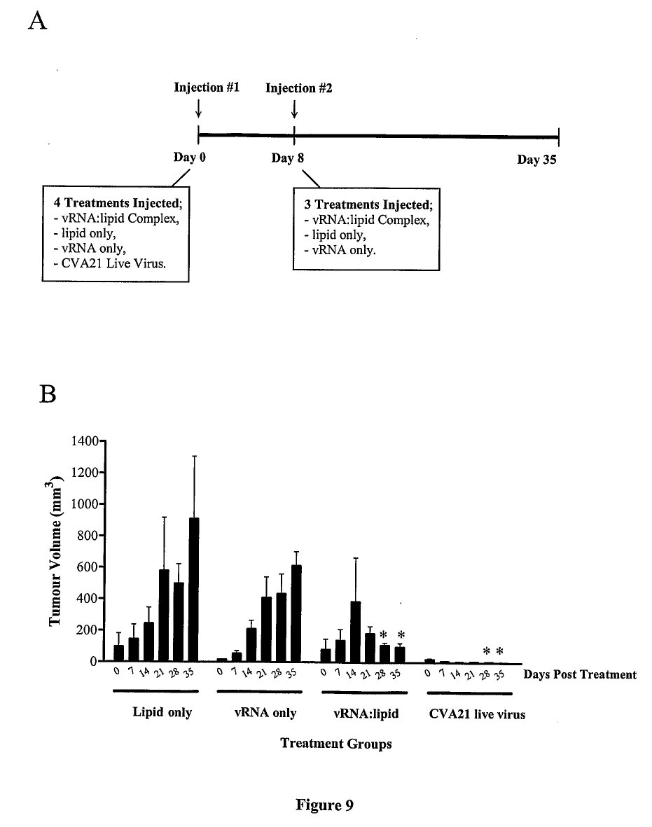

Two intersecting measurements of subcutaneous human melanoma tumours in NOD-SCID mice were estimated externally using digital calipers throughout the observation period and tumour volumes were calculated using the formula for a spheroid. Mice treated with lipid only developed large, nodular tumours. In contrast, all mice treated with CVA21 vRNA:lipid complex that were serum positive for infectious CVA21 (viremic) exhibited dramatic reduction in tumour volume over the examination period. Similarly, the tumour volumes of all CVA21 live virus treated mice were significantly reduced (FIG. 9). The average tumour volumes of mice treated with only vRNA did not decline, however, the tumour volumes for individual CVA21 viremic mice were reduced (see herein above).

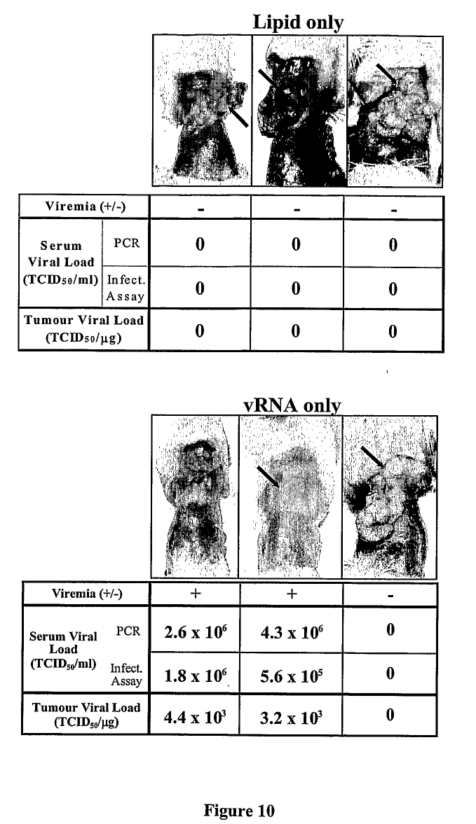

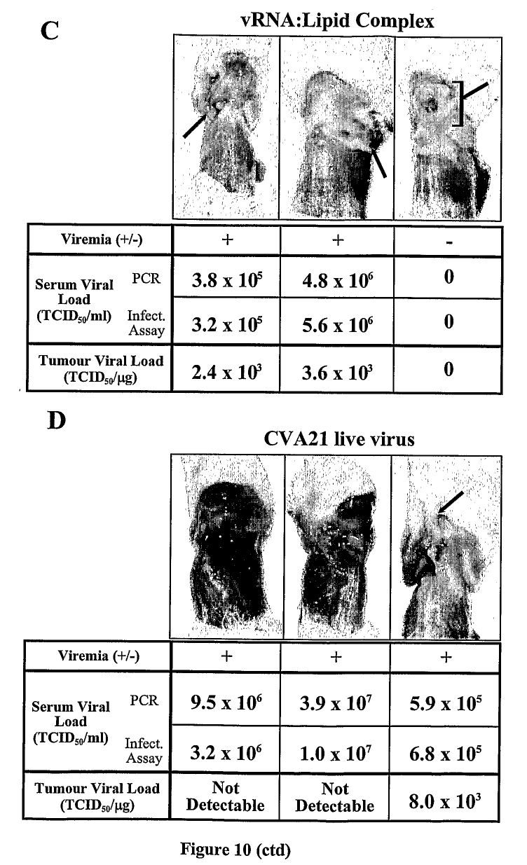

Macroscopic tumour characteristics were examined post mortem by dissection of the fur and skin from the back of each mouse. Mice in the lipid only group exhibited expansive, nodular and highly vascularised tumours (FIG. 10A). Of the mice treated with vRNA only, those that were viremic exhibited considerably smaller, less vascularised tumours compared to those mice that were not viremic (FIG. 10B). Similarly, the tumour burdens of viremic vRNA:lipid complex treated mice were dramatically reduced compared to both the non-viremic mouse in the group and the lipid only control mice (FIG. 10C). Tumours were also dramatically reduced in all mice treated with CVA21 live virus treated mice with some tumours undetectable upon dissection (FIG. 10D).

5.2. Levels of Infectious CVA21 in Lysates of Melanoma Xenografts

Homogenised tumour sections from selected mice each treatment group, isolated post mortem were tested for levels of infectious CVA21 by lytic cell infectivity assays on SK-MeI-28 cells. Cell monolayers in 96 well plates were inoculated with 10-fold dilutions of homogenate supernatants and evaluated microscopically for CPE following 72 hours incubation at 37° C. in 5% CO₂. Tumour viral load (TCID₅₀/μg) was calculated using the method of Reed and Muench (ibid). Tumour homogenates of all viremic mice exhibited substantial levels of infectious virus within the tumour tissue (up to 8×10³ TCID₅₀/μg of tumour). All tumour sections tested from non-viremic mice did not contain infectious CVA21 (FIG. 10).

### Discussion

Overview

The clinical application of viral oncolytic therapy could potentially be improved by providing methods which address issues such as bio-safety and costing considerations associated with the large-scale production, storage, distribution and administration of infectious virus. As demonstrated herein the administration of viral RNA instead of live virus provides an alternative to the administration of live virus. Large-scale in vitro production of viral RNA transcripts and or the potential use of infectious cDNA clones of oncolytic viruses represents an important novel direction towards the commercialization of viral oncolytic therapy. The use of viral RNA or cDNA clones of oncolytic viruses could significantly improve the safety of viral oncolytic therapy in respect to storage distribution and administration, as this substrate is only infectious once it enters from the cell surface to the cytoplasm.

Current treatments for malignant melanoma are limited if not diagnosed early and as most conventional radio- and chemotherapies offer little control of metastatic spread, a more efficient, selective therapy is required. CVA21 has proven to be an effective oncolytic agent against human melanoma cells in vitro and melanoma xenografts established in immuno-compromised mice in vivo (Shafren et al (2004; ibid).

Taken together, the results presented herein support demonstrate that introduction of liposome-complexed CVA21 viral RNA into the cell cytoplasm results in infectious progeny virus. Additionally, such progeny virus are demonstrated herein to elicit a reduction in human melanoma tumour volume in vivo.

In Vitro Production of Infectious Progeny CVA21

The delivery of CVA21 viral RNA using a cationic liposome (CVA21 vRNA:lipid complex) resulted in the production of infectious progeny virus in vitro and lytic infection of melanoma cells. Progeny virus could also be produced in cells normally not susceptible to natural CVA21 infection, due to the lack of CVA21 cell surface receptor expression. This infection, however, was limited to one round of replication, as progeny virus, when passaged, was unable to infect the same cell type. This result suggests that progeny CVA21, produced by delivery of CVA21 vRNA:lipid complex, retains its specificity for cells expressing high levels of ICAM-1 and DAF. In contrast, the delivery of naked CVA21 vRNA only, without a lipid carrier was unable to mediate progeny virus production in vitro, although was associated in some animals with a reduction in tumor volume, thereby demonstrating the potential for use of naked viral RNA.

Compared to the infection with CVA21 live virus, vRNA:lipid complex transfection required a longer time to induce similar levels of microscopic CPE, (approximately 12 hours longer for complete CPE). The morphology of cell death was, however, identical to infection with live CVA21. This lag in CPE progression is most likely due to the time taken for liposome complexes to randomly associate with a cell, which in this instance is based on charge rather than specific receptor interactions. Theoretically, once the vRNA had been translocated into the cell cytoplasm, viral replication should occur at the same rate as in natural infection with live CVA21 virions.

In Vivo Production of Infectious CV21

Intra-tumoural delivery of CVA21 vRNA:lipid complex to a human melanoma xenograft in NOD-SCID mice resulted in the production of infectious CVA21 progeny in both the serum and tumour tissue (4/5-treated mice). Two separate injections of vRNA:lipid complex, vRNA only and lipid only were given at days 0 and 8, however, no viremia was detected at days 0, 2, 5 or 9 following initial treatment, with CVA21 vRNA detected for the first time at day 16. This delay in viremia may be attributed to poor transfection efficiency of the liposome, as interactions with serum proteins and other factors have been reported to interfere with the function of liposomes in vivo (Tandia, B.-M., et al., Identification of human plasma proteins that bind to cationic lipid/DNA complex and analysis of their effects on transfection efficiency: implications for intravenous gene transfer. Molecular Therapy, 2003. 8(2):264-273). The necrotic tumour environment, typically contains large numbers of serum proteins, therefore liposome complex formation may be severely disrupted. Other factors which may have contributed to delay of viremia include; unfavourable nucleic acid:liposome charge ratio, injection volume and injection accuracy. At the initial injection day (day 0) tumours were relatively small, and efficacy of direct intra-tumoural injection may have been compromised. On day 8, however, the tumour volumes had increased and were much more accessible for direct injection. Additional testing at time-points at days 9-16 may have revealed data supporting a faster response, but ethical consent for additional serum samples was not authorized. In contrast, mice injected with CVA21 live virus exhibited detectable levels of vRNA in the serum as early as 2 hours post-injection, indicating systemic spill over from the tumour.

Contrary to in vitro results, the intra-tumoural delivery of naked vRNA resulted in the production of infectious progeny CVA21 in vivo in 3/4 treated mice. This progeny virus production was not detected in the serum until day 16 and occurred at similar levels to the virus produced by mice injected with vRNA:lipid complex (4/5 mice). Progeny virus from vRNA only injected mice was also detected in post mortem tumour tissue, a result that was somewhat unexpected as no viral replication could be detected in vitro when exposed to the same vRNA inoculum. This finding may be attributed to the differences between the intra-tumoural environment and the free-flowing cell culture media. It would be reasonable to assume, that within an in vitro environment, RNases and other cellular factors within the media could degrade the naked vRNA, and furthermore, there would be no mechanism to overcome the electrostatic forces between the vRNA and the membrane to enter the cell. The in vivo environment may be more active, with a small (50 μl) bolus injection trapped within the tumour tissue leading to more interaction between the tumour cells and the vRNA, possibly facilitating vRNA uptake by the tumour cells.

In Vivo Oncolytic Action of Infectious Progeny CVA21

Following delivery of CVA21 vRNA:lipid complex, the oncolytic action of progeny CVA21 resulted in reduction of the tumour burden of immuno-compromised mice bearing established subcutaneous human melanoma xenografts. On day 28 and 35, group average tumour volumes of the vRNA:lipid treated mice was significantly reduced compared to the group treated only with lipid on days 28 and 35 (p<0.05). Similarly, mice treated with live CVA21 virus also exhibited significant reduction in tumour volume on days 28 and 35 (p<0.05) in comparison to the group treated only with lipid. Although 3/4 mice treated with vRNA alone were viremic, no statistically significant reduction in the average tumour volume was evident in this group when compared to the group treated only with lipid.

Post mortem examination of tumours confirmed the hypothesis that infectious serum CVA21 resulted in the reduction of tumour burden of human melanoma xenografts. All viremic mice, regardless of the treatment regime they received, exhibited substantial reductions in tumour volume. Conversely, those mice that remained non-viremic, regardless of treatment, supported the growth of large nodular tumours. Taken together this data supports the finding that the presence of CVA21 viremia in NOD-SCID mice resulted in drastically reduced tumour burdens of established subcutaneous human melanoma xenografts.

### CONCLUSIONS

Malignant melanoma is one of the most commonly occurring cancers in Australia with little to no effective treatment available if not diagnosed and surgically excised early. Viral oncolytic therapy with CVA21 offers a novel treatment avenue for the systemic treatment and control of malignant melanoma progression. The administration of infectious viral RNA may prove advantageous to the usage of intact live virus if bio-safety and costing issues would limit the widespread administration of CVA21 viral oncolytic therapy.

This study investigated the efficiency of liposome-mediated delivery of CVA21 viral RNA to produce infectious progeny CVA21 in vitro and the ability of the progeny virus to lyse human melanoma cells and reduce established melanoma xenograft tumour burdens in vivo. The delivery of liposome-complexed CVA21 viral RNA resulted in production of infectious progeny virus both in vitro and in vivo. In addition, administration of viral RNA alone in vivo also resulted in infectious virus production. Infectious progeny virus, regardless of the treatment administered, was able to infect and reduce the tumour burden of human melanoma xenografts in immuno-compromised mice.

1. A method for treating a neoplasm in a mammal requiring said treatment, the method comprising administering to the mammal an effective amount of a nucleic acid molecule comprising an isolated viral polynucleotide sequence derived from a Picornavirus under conditions which result in virus-mediated oncolysis of one or more cells of the neoplasm.

2. The method according to claim 1, wherein the neoplasm is selected from the group consisting of prostate cancer, breast cancer, ovarian cancer, lymphoid cancer, leukemia, brain cancer, lung cancer, colorectal cancer, thyroid cancer, renal cancer, adrenal cancer, liver cancer, stomach cancer, intestinal cancer and melanoma.

3. The method according to claim 1, wherein the nucleic acid molecule is selected from single stranded RNA or complementary DNA (cDNA) comprising a sequence derived from the Picornavirus.

4. The method according to claim 1, wherein the nucleic acid molecule comprises a viral genome or a portion thereof capable of eliciting a lytic infection when administered to a cell.

5. The method according to claim 1, wherein the nucleic acid molecule is synthetic viral RNA.

6. The method according to claim 1, wherein the nucleic acid molecule is derived from a Picornavirus which recognises at least one of a cell adhesion molecule of the immunoglobulin superfamily and a complement regulatory protein for infectivity of a cell.

7. The method according to claim 1, wherein the nucleic acid molecule is derived from a Picornavirus which recognises α₂β₁ for infectivity of a cell.

8. The method according to claim 1, wherein the nucleic acid molecule is derived from a Picornavirus which recognises at least one of ICAM-1 and DAF for infectivity of a cell.

9. The method according to claim 1, wherein the nucleic acid molecule is derived from a Picornavirus capable of lytically infecting or inducing apoptosis in a cell substantially in the absence of ICAM-1.

10. The method according to claim 1, wherein the nucleic acid molecule is derived from a Picornavirus capable of lytically infecting or inducing apoptosis in a cell through DAF on the cell.

11. The method according to claim 1, wherein the nucleic acid molecule is derived from a Coxsackie virus selected from the group consisting of CVA13, CVA15, CVA18 and CVA21.

12. The method according to claim 1, wherein the nucleic acid molecule is derived from an Echovirus selected from the group consisting of EV1, EV7, EV8 and EV22.

13. The method according to claim 1, wherein the polynucleotide sequence comprises an alteration in one or more capsid proteins compared with wild-type wherein the alteration enhances cell selectivity and or neoplasm targeting of a virus comprising the alteration.

14. The method according to claim 1, wherein the polynucleotide sequence comprises a Coxsackie virus nucleic acid sequence comprising one or more mutations of a coat protein.

15. The method according to claim 14, wherein the mutations of the coat protein comprises one or more mutations selected from the group consisting of VP3 R96H, VP3 E101A, VP3 A239S, VP2 S164L and VP2 V209 or corresponding conservative variants thereof.

16. The method according to claim 1, wherein the nucleic acid molecule is administered as a formulation comprising vRNA and lipid, such as a cationic lipid.

17. The method according to claim 16, wherein the formulation further comprises a ligand which recognises and interacts with a tumour-specific marker.

18. The method according to claim 16, wherein the formulation further comprises an antibody which recognises a tumour antigen, such as a monoclonal antibody which recognises DAF, ICAM-1, α₂β₁, or MAGE.

19. The method according to claim 1, wherein the nucleic acid molecule is administered by one or more methods selected from direct injection into a neoplasm, orally and systemically.

20. The method according to claim 1, wherein the method further comprises administration of one or more immunosuppressants to the mammal.

21. The method according to claim 1, wherein the mammal is a human.

22. A method for treating melanoma in a human requiring said treatment, the method comprising administering to the human an effective amount of a formulation comprising vRNA and lipid, wherein the vRNA comprises RNA isolated from one or more viruses selected from the group consisting of CVA13, CVA15, CVA18, CVA21, EV1, EV7, EV8 and EV22, and variants CVA21 #272101, CVA21 #275238, and CVA21 #272598, and CVA21-DAFv under conditions which result in virus-mediated oncolysis of one or more cells of the melanoma.

23. The method according to claim 22, wherein administration is direct injection of the formulation into one or more melanoma(s) of the human.

24. The method according to claim 22, wherein the formulation further comprises a ligand which recognises and interacts with a tumour-specific marker.

25. The method according to claim 22, wherein the formulation further comprises an antibody which recognises a tumour antigen, such as a monoclonal antibody which recognises DAF, ICAM-1, α₂β₁, or MAGE.

26. Use of a nucleic acid molecule comprising an isolated viral nucleic acid sequence derived from a Picornavirus for the preparation of a medicament for the treatment of a neoplasm in a mammal.

27. A pharmaceutical composition comprising a nucleic acid molecule comprising an isolated viral polynucleotide sequence derived from a Picornavirus together with a pharmaceutically acceptable vehicle, diluent or carrier, wherein administration of the pharmaceutical composition to a neoplasm results in virus-mediated oncolysis of one or more cells of the neoplasm.

28. The composition according to claim 27, wherein the nucleic acid molecule is derived from one or more viruses selected from the group consisting of CVA13, CVA15, CVA18, CVA21, EV1, EV7, EV8 and EV22 and variants CVA21 #272101, CVA21 #275238, and CVA21 #272598, and CVA21-DAFv.

29. The composition according to claim 27, further comprising one or more lipids, such as a cationic lipid.

30. The composition according to claim 29, wherein said composition comprises a vRNA:lipid complex.

31. The composition according to claim 30, wherein said composition comprises a CVA21:lipid complex.

32. The composition according to claim 27, further comprising a ligand which recognises and interacts with a tumour-specific marker.

33. The composition according to claim 27, further comprising an antibody which recognises a tumour antigen.

34. The composition according to claim 33, wherein said antibody is a monoclonal antibody which recognises DAF, ICAM-1, α₂β₁, or MAGE.


SEQUENCE LISTING (3 sequences)

SEQ ID NO: 1 - DNA, 22 bp, Artificial Sequence
  Primer sequence used for RT-PCR of CVA21 Kuykendall Strain VP3 region
gagctaaaccaccaaccaatcg

SEQ ID NO: 2 - DNA, 19 bp, Artificial Sequence
  Primer sequence used for RT-PCR of CVA21 Kuykendall Strain VP3 region
cggtgcaaccatggaacaa

SEQ ID NO: 3 - DNA, 18 bp, Artificial Sequence
  Probe sequence used for RT-PCR of CVA21 Kuykendall Strain VP3 region
cacacacatcatctggga